SEQUENCE LISTING (112 sequences)

SEQ ID NO: 1 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAGRQVAWLGNWGQGTLVTVSS

SEQ ID NO: 2 - protein, 5 aa, synthetic construct
SYAMS

SEQ ID NO: 3 - protein, 17 aa, synthetic construct
AISGSGGSTYYADSVKG

SEQ ID NO: 4 - protein, 8 aa, synthetic construct
RQVAWLGN

SEQ ID NO: 5 - protein, 107 aa, synthetic construct
DIVMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVDIK

SEQ ID NO: 6 - protein, 11 aa, synthetic construct
RASQGISSWLA

SEQ ID NO: 7 - protein, 7 aa, synthetic construct
AASSLQS

SEQ ID NO: 8 - protein, 9 aa, synthetic construct
QQANSFPLT

SEQ ID NO: 9 - protein, 116 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAGGANGFDPWGQGTLVTVSS

SEQ ID NO: 10 - protein, 5 aa, synthetic construct
SYAMS

SEQ ID NO: 11 - protein, 17 aa, synthetic construct
AISGSGGSTYYADSVKG

SEQ ID NO: 12 - protein, 7 aa, synthetic construct
GANGFDP

SEQ ID NO: 13 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGLVFGGGTKLTVL

SEQ ID NO: 14 - protein, 14 aa, synthetic construct
TGSSSNIGAGYDVH

SEQ ID NO: 15 - protein, 7 aa, synthetic construct
GNSNRPS

SEQ ID NO: 16 - protein, 11 aa, synthetic construct
QSYDSSLSGLV

SEQ ID NO: 17 - protein, 120 aa, synthetic construct
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCAKDQRGGLYGMDVWGQGTLVTVSS

SEQ ID NO: 18 - protein, 5 aa, synthetic construct
SYGMH

SEQ ID NO: 19 - protein, 17 aa, synthetic construct
VISYDGSNKYYADSVKG

SEQ ID NO: 20 - protein, 11 aa, synthetic construct
DQRGGLYGMDV

SEQ ID NO: 21 - protein, 111 aa, synthetic construct
QSVLTQPPSASGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVL

SEQ ID NO: 22 - protein, 14 aa, synthetic construct
TGSSSNIGAGYDVH

SEQ ID NO: 23 - protein, 7 aa, synthetic construct
GNSNRPS

SEQ ID NO: 24 - protein, 11 aa, synthetic construct
QSYDSSLSGWV

SEQ ID NO: 25 - protein, 116 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSGFDYWGQGTLVTVSS

SEQ ID NO: 26 - protein, 5 aa, synthetic construct
SYYMH

SEQ ID NO: 27 - protein, 17 aa, synthetic construct
IINPSGGSTSYAQKFQG

SEQ ID NO: 28 - protein, 7 aa, synthetic construct
GGSGFDY

SEQ ID NO: 29 - protein, 110 aa, synthetic construct
SYVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGWVFGGGTKLTVL

SEQ ID NO: 30 - protein, 13 aa, synthetic construct
SGSSSNIGSNYVY

SEQ ID NO: 31 - protein, 7 aa, synthetic construct
SNNQRPS

SEQ ID NO: 32 - protein, 11 aa, synthetic construct
AAWDDSLSGWV

SEQ ID NO: 33 - protein, 118 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAIPGDYGSSDPWGQGTLVTVSS

SEQ ID NO: 34 - protein, 5 aa, synthetic construct
SYAMS

SEQ ID NO: 35 - protein, 17 aa, synthetic construct
AISGSGGSTYYADSVKG

SEQ ID NO: 36 - protein, 9 aa, synthetic construct
PGDYGSSDP

SEQ ID NO: 37 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKLTVL

SEQ ID NO: 38 - protein, 14 aa, synthetic construct
TGSSSNIGAGYDVH

SEQ ID NO: 39 - protein, 7 aa, synthetic construct
GNSNRPS

SEQ ID NO: 40 - protein, 11 aa, synthetic construct
QSYDSSLSGYV

SEQ ID NO: 41 - protein, 125 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDVTYYYDSSGSPDFDYWGQGTLVTVSS

SEQ ID NO: 42 - protein, 5 aa, synthetic construct
SYGMH

SEQ ID NO: 43 - protein, 17 aa, synthetic construct
VISYDGSNKYYADSVKG

SEQ ID NO: 44 - protein, 16 aa, synthetic construct
DVTYYYDSSGSPDFDY

SEQ ID NO: 45 - protein, 107 aa, synthetic construct
DIQLTQSPSSLSASVGDRVTITCRASQGISNYLAWFQQKPGKAPKSLIYAASSLQSGVPSKFSGSGSGTDFTLTISSLQPEDFATYYCQQYNSYPYSFGQGTKVEIK

SEQ ID NO: 46 - protein, 11 aa, synthetic construct
RASQGISNYLA

SEQ ID NO: 47 - protein, 7 aa, synthetic construct
AASSLQS

SEQ ID NO: 48 - protein, 9 aa, synthetic construct
QQYNSYPYS

SEQ ID NO: 49 - protein, 117 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAGYTGGAFDIWGQGTMVTVSS

SEQ ID NO: 50 - protein, 5 aa, synthetic construct
SYAMS

SEQ ID NO: 51 - protein, 17 aa, synthetic construct
AISGSGGSTYYADSVKG

SEQ ID NO: 52 - protein, 8 aa, synthetic construct
YTGGAFDI

SEQ ID NO: 53 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIK

SEQ ID NO: 54 - protein, 11 aa, synthetic construct
RASQGISSWLA

SEQ ID NO: 55 - protein, 7 aa, synthetic construct
AASSLQS

SEQ ID NO: 56 - protein, 9 aa, synthetic construct
QQANSFPLT

SEQ ID NO: 57 - protein, 119 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKPLRFLGDYGGWGQGTLVTVSS

SEQ ID NO: 58 - protein, 5 aa, synthetic construct
SYAMS

SEQ ID NO: 59 - protein, 17 aa, synthetic construct
AISGSGGSTYYADSVKG

SEQ ID NO: 60 - protein, 10 aa, synthetic construct
PLRFLGDYGG

SEQ ID NO: 61 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIK

SEQ ID NO: 62 - protein, 11 aa, synthetic construct
QASQDISNYLN

SEQ ID NO: 63 - protein, 7 aa, synthetic construct
DASNLET

SEQ ID NO: 64 - protein, 9 aa, synthetic construct
QQYDNLPLT

SEQ ID NO: 65 - protein, 114 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCATRQFDYWGQGTLVTVSS

SEQ ID NO: 66 - protein, 5 aa, synthetic construct
SYYMH

SEQ ID NO: 67 - protein, 17 aa, synthetic construct
IINPSGGSTSYAQKFQG

SEQ ID NO: 68 - protein, 5 aa, synthetic construct
RQFDY

SEQ ID NO: 69 - protein, 110 aa, synthetic construct
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAAVFGGGTKVTVL

SEQ ID NO: 70 - protein, 13 aa, synthetic construct
SGSSSNIGNNYVS

SEQ ID NO: 71 - protein, 7 aa, synthetic construct
ENNKRPS

SEQ ID NO: 72 - protein, 11 aa, synthetic construct
GTWDSSLSAAV

SEQ ID NO: 73 - protein, 118 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGVYYFDYWGQGTLVTVSS

SEQ ID NO: 74 - protein, 5 aa, synthetic construct
SYAMS

SEQ ID NO: 75 - protein, 17 aa, synthetic construct
AISGSGGSTYYADSVKG

SEQ ID NO: 76 - protein, 9 aa, synthetic construct
DLGVYYFDY

SEQ ID NO: 77 - protein, 107 aa, synthetic construct
DIVMTQSPSFLSASAGDRVTITCRASQGISSYLAWYQQKPGKGPKLLIYAASTLQSGVPSRFSGSGSGTEFTLTISSLQPEDFATYYCQQVNSYPLTFGPGTKVDIK

SEQ ID NO: 78 - protein, 11 aa, synthetic construct
RASQGISSYLA

SEQ ID NO: 79 - protein, 7 aa, synthetic construct
AASTLQS

SEQ ID NO: 80 - protein, 9 aa, synthetic construct
QQVNSYPLT

SEQ ID NO: 81 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEPFYGMDVWGQGTLVTVSS

SEQ ID NO: 82 - protein, 5 aa, synthetic construct
SYAMS

SEQ ID NO: 83 - protein, 17 aa, synthetic construct
AISGSGGSTYYADSVKG

SEQ ID NO: 84 - protein, 8 aa, synthetic construct
EPFYGMDV

SEQ ID NO: 85 - protein, 107 aa, synthetic construct; identical to SEQ ID NO: 53

SEQ ID NO: 86 - protein, 11 aa, synthetic construct
RASQGISSWLA

SEQ ID NO: 87 - protein, 7 aa, synthetic construct
AASSLQS

SEQ ID NO: 88 - protein, 9 aa, synthetic construct
QQANSFPLT

SEQ ID NO: 89 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPPGSYYVWDYWGQGTLVTVSS

SEQ ID NO: 90 - protein, 5 aa, synthetic construct
SYAMS

SEQ ID NO: 91 - protein, 17 aa, synthetic construct
AISGSGGSTYYADSVKG

SEQ ID NO: 92 - protein, 10 aa, synthetic construct
PPGSYYVWDY

SEQ ID NO: 93 - protein, 107 aa, synthetic construct
DIVMTQSPSTLSASVGDRVTITCRASQSISSWLAWYQQKPGKAPKLLIYKASSLESGVPSRFSGSGSGTEFTLTISSLQPDDFATYYCQQYNSYPLTFGGGTKVEIK

SEQ ID NO: 94 - protein, 11 aa, synthetic construct
RASQSISSWLA

SEQ ID NO: 95 - protein, 7 aa, synthetic construct
KASSLES

SEQ ID NO: 96 - protein, 9 aa, synthetic construct
QQYNSYPLT

SEQ ID NO: 97 - protein, 119 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKTAGSYYILDYWGQGTLVTVSS

SEQ ID NO: 98 - protein, 5 aa, synthetic construct
SYAMS

SEQ ID NO: 99 - protein, 17 aa, synthetic construct
AISGSGGSTYYADSVKG

SEQ ID NO: 100 - protein, 10 aa, synthetic construct
TAGSYYILDY

SEQ ID NO: 101 - protein, 107 aa, synthetic construct
DIQLTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVDIK

SEQ ID NO: 102 - protein, 11 aa, synthetic construct
RASQGISNYLA

SEQ ID NO: 103 - protein, 7 aa, synthetic construct
AASSLQS

SEQ ID NO: 104 - protein, 9 aa, synthetic construct
QQYNSYPLT

SEQ ID NO: 105 - protein, 124 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKPLTAGYYYGMVAFDIWGQGTMVTVSS

SEQ ID NO: 106 - protein, 5 aa, synthetic construct
SYAMS

SEQ ID NO: 107 - protein, 17 aa, synthetic construct
AISGSGGSTYYADSVKG

SEQ ID NO: 108 - protein, 15 aa, synthetic construct
PLTAGYYYGMVAFDI

SEQ ID NO: 109 - protein, 107 aa, synthetic construct
DIVMTQSPSFLSASVGDRVTITCRASQGISSYLAWYQQKPGKAPKLLIYAASTLQSGVPSRFSGSGSGTEFTLTISSLQPEDFATYYCQQLNSYPITFGQGTKLEIK

SEQ ID NO: 110 - protein, 11 aa, synthetic construct
RASQGISSYLA

SEQ ID NO: 111 - protein, 7 aa, synthetic construct
AASTLQS

SEQ ID NO: 112 - protein, 9 aa, synthetic construct
QQLNSYPIT

(19) United States
(12) Patent Application Publication
(10) Pub. No.: US 2026/0151520 A1
(43) Pub. Date: Jun. 4, 2026

(54) CLAUDIN 18.2-TARGETED COMPOUNDS AND USES THEREOF

(71) Applicant: Fusion Pharmaceuticals Inc., Hamilton (CA)

(72) Inventors: Natalie Grinshtein, Bothell, WA (US); John Richard Forbes, Burlington (CA); Sonal Patel, Hamilton (CA); Ian R. Duffy, Oakville (CA); Thomas I. Kostelnik, Hamilton (CA)

(21) Appl. No.: 18/703,994

(22) PCT Filed: Oct. 25, 2022
(86) PCT No.: PCT/CA2022/051573

§ 371 Date: Apr. 23, 2024

Related U.S. Application Data
(60) Provisional application No. 63/271,634, filed on Oct. 25, 2021.

(51) Int. Cl. A61K 51/10; A61P 35/00; C07K 16/28

(52) U.S. Cl. CPC A61K 51/1027; A61K 51/1096; A61P 35/00; C07K 16/28; A61K 2121/00

(57) ABSTRACT

Compounds, e.g., radioimmunoconjugates including a chelating moiety or a metal complex thereof, a linker, and an Claudin 18.2 targeting moiety. Pharmaceutical compositions of such compounds and methods of treatment for conditions, e.g., cancer, using such pharmaceutical compositions.

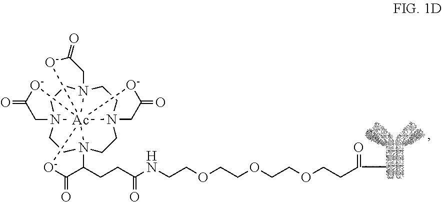

### RELATED APPLICATIONS

The present application claims priority to U.S. Provisional Patent Application No. 63/271,634, filed Oct. 25, 2021 the entire contents of which are hereby incorporated by reference for all purposes.

### SEQUENCE LISTING

The instant application contains a Sequence Listing which has been submitted electronically in XML format and is hereby incorporated by reference in its entirety. Said XML file, created on Oct. 24, 2022, is named 20221024_FPI_029WO.xml and is 99 kilobytes in size.

### BACKGROUND

Stomach, esophageal (gastroesophageal; GE) and pancreatic cancers are malignancies with highest unmet medical need. Gastric cancer is the second leading cause of cancer death worldwide while pancreatic cancer is currently fourth leading cause of cancer-related deaths in United States. Majority of patients with gastric cancer are often diagnosed in the advanced stage of the disease, and treatment mostly entails palliative chemotherapy conferring median PFS (progression free survival) of 5 to 7 months and median OS (overall survival) of 9 to 11 months at the best. On the other hand, pancreatic cancer has extremely poor prognosis with only about 9% of 5-year survival rate.

The lack of a major benefit from the newer generation combination chemotherapy regimens for these cancers indicates that there is major need for newer targeted therapies. One such promising approach being tested in clinic is antibody drug conjugates (ADCs) that deliver cytotoxic payload attached antibody specifically to cancer cells. However, ADCs have their own limitation such as off-target payload related adverse events limiting the maximum tolerated dose. Also, ADCs are efficacious only if the cells express high levels of cell surface tumor associated antigens (TAAs). This calls for the need of developing more promising targeted modalities such as targeted radiotherapy.

Claudins are a family of tight junction proteins with Claudin 18 splice variant 2 (Claudin 18.2 (CLDN18.2)) being one of its members. Claudins mainly form paracellular barrier and pores and regulate the transport of substances by determining the permeability of the tight junctions. The expression of CLDN18.2 is not detectable in normal healthy human tissues except for stomach. However, it is aberrantly expressed at significant levels about 60-90% in gastroesophageal cancer as well as its metastases and pancreatic cancer making it an attractive therapeutic target. Currently, chimeric IgG1 antibody, IMAB362, directed against CLDN18.2 is in Phase III clinical trial in combination with mFOLFOX6 (NCT03504397) and CAPOX (NCT03653507) chemotherapy in patients with locally advanced unresectable or metastatic gastric and gastroesophageal (GEJ) cancer. Delivering CLDN18.2 therapeutic radionuclides may produce enhanced efficacy at substantially lower doses than that of an antibody alone.

Thus, there remains a need for improved therapeutics (e.g., cancer therapeutics) that can target CLDN18.2.

### SUMMARY

The present disclosure relates to compounds that target Claudin 18.2 (CLDN18.2), pharmaceutical compositions thereof, and methods of treating cancer using such pharmaceutical compositions. In certain embodiments, provided compounds exhibit an increased excretion rate (e.g., after being administered to a mammal) compared to some currently known radiotherapeutics, while still maintaining therapeutic efficacy. In some embodiments, a faster excretion may limit off-target toxicities by limiting the amount of time that the compound stays in a subject. Thus, in some embodiments, provided compounds exhibit reduced off-target toxicities.

In one aspect, provided are compounds comprising the following structure, or pharmaceutically acceptable salts thereof.

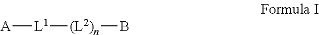

wherein
A is a chelating moiety or a metal complex thereof,
B is a targeting moiety that is capable of binding to Claudin 18.2 (CLDN18.2) or a fragment thereof;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

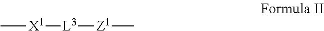

wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀ alkyl or optionally substituted C₁-C₅₀ heteroalkyl; and
Z¹ is —CH₂— #, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

In certain embodiments, variable A in Formula I is a chelating moiety selected from the group consisting of DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaoxacyclohexadecane-1,4,7,10,13,16-hexaacetic acid), PEPA (1,4,7,10,13-pentaazacyclopentadecane-N,N′,N″,N′″,N″″-pentaacetic acid), H₄ octapa (N,N′-bis(6-carboxy-2-pyridylmethyl)-ethylenediamine-N,N′-diacetic acid), H₂ dedpa (1,2-[[6-(carboxy)-pyridin-2-yl]-methylamino]ethane), H₆ phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″,N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

In some embodiments, the compound of Formula I is represented by:

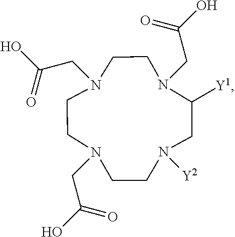

wherein Y¹ is —CH₂OCH₂(L²)ₙ-B, C═O(L²)ₙ-B, or C═S(L²)ₙ-B and Y² is —CH₂CO₂H; or
wherein Y¹ is H and Y² is L¹-(L²)ₙ-B. In certain embodiments, Y¹ is H.

In some embodiments, L¹ is

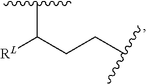

and R^{L} is hydrogen or —CO₂H.

In certain embodiments, X¹ is —C(O)NR¹—* or —NR¹C(O)—*, “*” indicating the attachment point to L³, and R¹ is H.

In certain embodiments, Z¹ is —CH₂—.

In some embodiments, L³ comprises (CH₂CH₂O)₂₋₂₀. In some embodiments, L³ is (CH₂CH₂O)ₘ(CH₂)_{w}, wherein m and w are each independently an integer between 0 and 10 (inclusive), and at least one of m and w is not 0.

In some embodiments, the metal complex comprises a metal selected from the group consisting of Bi, Pb, Y, Mn, Cr, Fe, Co, Zn, Ni, Tc, In, Ga, Cu, Re, a lanthanide, and an actinide. In some embodiments, the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, variable A is a metal complex of a chelating moiety. In some such embodiments, the metal complex comprises a radionuclide. In some embodiments, the radionuclide is an alpha emitter, e.g., an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In some embodiments, the radionuclide is ⁶⁸Ga, ¹¹¹n, ¹⁷⁷Lu, or ²²⁵Ac. In some embodiments, the radionuclide is ²²⁵Ac or a progeny thereof.

In some embodiments, A-L- in Formula I comprises one of the following structures, or a metal complex thereof:

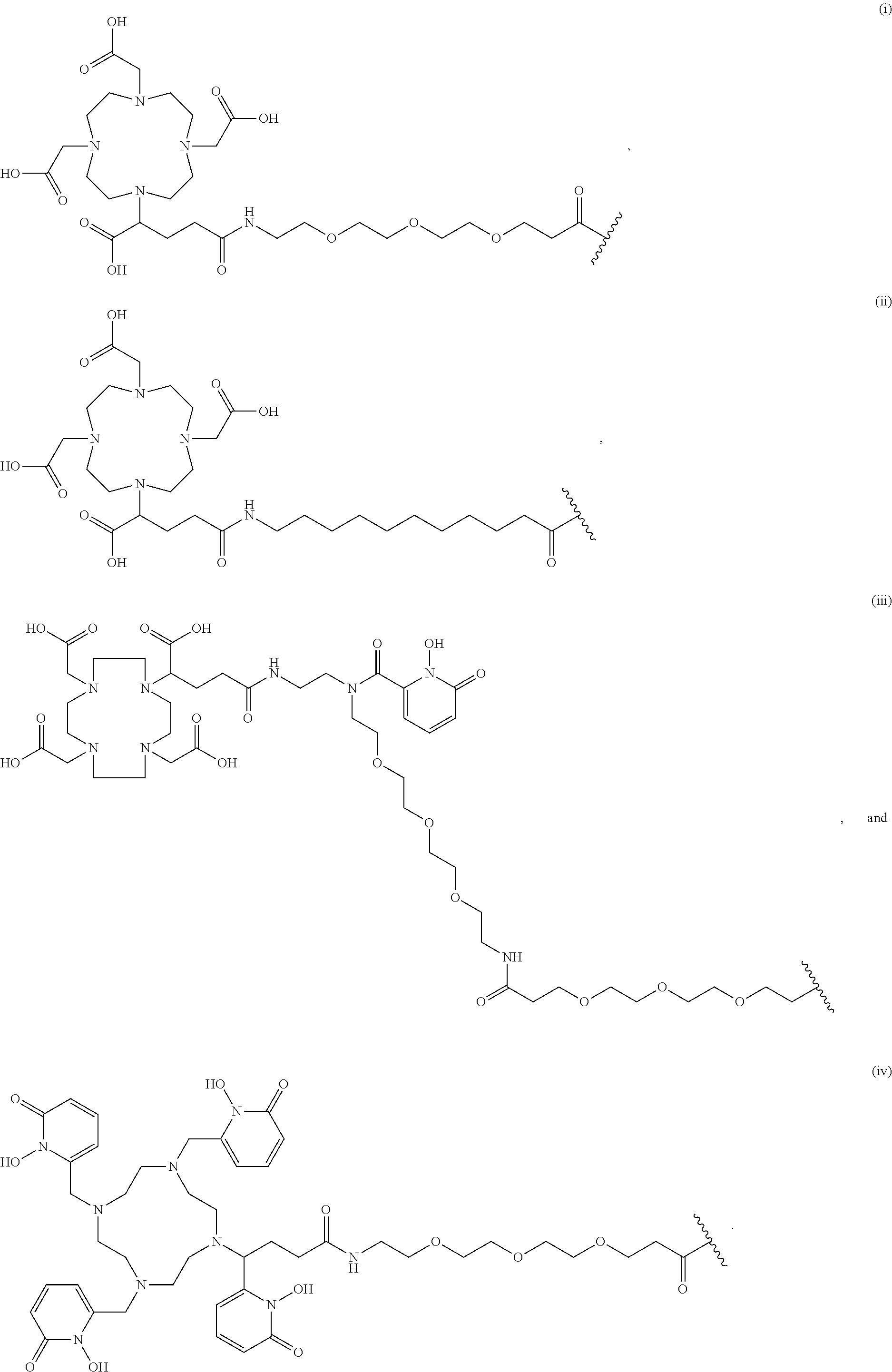 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O ; *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, the compound or a pharmaceutically acceptable salt thereof comprises the following structure, or a metal complex thereof:

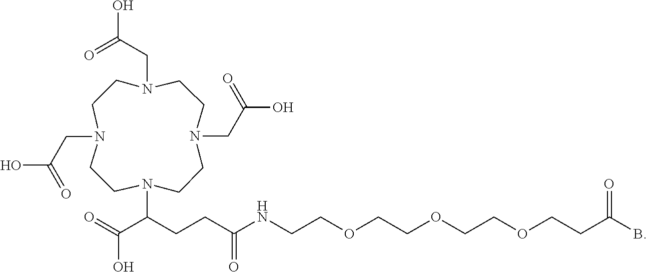

In some embodiments, the targeting moiety comprises an antibody or antigen-binding fragment thereof that specifically binds to CLDN18.2 or a fragment thereof.

In some embodiments, the antibody or antigen-binding fragment thereof comprises:
(a)(i) a heavy chain variable domain (VH) comprising complementarity-determining regions: CDR-H1 comprising the amino acid sequence of SEQ ID NO: 2 or a sequence differing in 1 or 2 amino acids therefrom, CDR-H2 comprising the amino acid sequence of SEQ ID NO: 3 or a sequence differing in 1 or 2 amino acids therefrom, and CDR-H3 comprising the amino acid sequence of SEQ ID NO: 4 or a sequence differing in 1 or 2 amino acids therefrom; and
   (ii) a light chain variable domain (VL) comprising complementarity-determining regions: CDR-L1 comprising the amino acid sequence of SEQ ID NO: 6 or a sequence differing in 1 or 2 amino acids therefrom, CDR-L2 comprising the amino acid sequence of SEQ ID NO: 7 or a sequence differing in 1 or 2 amino acids therefrom, and CDR-L3 comprising the amino acid sequence of SEQ ID NO: 8 or a sequence differing in 1 or 2 amino acids therefrom;
(b)(i) a VH comprising complementarity-determining regions: CDR-H1 comprising the amino acid sequence of SEQ ID NO: 10 or a sequence differing in 1 or 2 amino acids therefrom, CDR-H2 comprising the amino acid sequence of SEQ ID NO: 11 or a sequence differing in 1 or 2 amino acids therefrom, and CDR-H3 comprising the amino acid sequence of SEQ ID NO: 12 or a sequence differing in 1 or 2 amino acids therefrom; and
   (ii) a VL comprising complementarity-determining regions: CDR-L1 comprising the amino acid sequence of SEQ ID NO: 14 or a sequence differing in 1 or 2 amino acids therefrom, CDR-L2 comprising the amino acid sequence of SEQ ID NO: 15 or a sequence differing in 1 or 2 amino acids therefrom, and CDR-L3 comprising the amino acid sequence of SEQ ID NO: 16 or a sequence differing in 1 or 2 amino acids therefrom;
(c)(i) a VH comprising complementarity-determining regions: CDR-H1 comprising the amino acid sequence of SEQ ID NO: 18 or a sequence differing in 1 or 2 amino acids therefrom, CDR-H2 comprising the amino acid sequence of SEQ ID NO: 19 or a sequence differing in 1 or 2 amino acids therefrom, and CDR-H3 comprising the amino acid sequence of SEQ ID NO: 20 or a sequence differing in 1 or 2 amino acids therefrom; and
   (ii) a VL comprising complementarity-determining regions: CDR-L1 comprising the amino acid sequence of SEQ ID NO: 22 or a sequence differing in 1 or 2 amino acids therefrom, CDR-L2 comprising the amino acid sequence of SEQ ID NO: 23 or a sequence differing in 1 or 2 amino acids therefrom, and CDR-L3 comprising the amino acid sequence of SEQ ID NO: 24 or a sequence differing in 1 or 2 amino acids therefrom;
(d)(i) a VH comprising complementarity-determining regions: CDR-H1 comprising the amino acid sequence of SEQ ID NO: 26 or a sequence differing in 1 or 2 amino acids therefrom, CDR-H2 comprising the amino acid sequence of SEQ ID NO: 27 or a sequence differing in 1 or 2 amino acids therefrom, and CDR-H3 comprising the amino acid sequence of SEQ ID NO: 28 or a sequence differing in 1 or 2 amino acids therefrom; and
   (ii) a VL comprising complementarity-determining regions: CDR-L1 comprising the amino acid sequence of SEQ ID NO: 30 or a sequence differing in 1 or 2 amino acids therefrom, CDR-L2 comprising the amino acid sequence of SEQ ID NO: 31 or a sequence differing in 1 or 2 amino acids therefrom, and CDR-L3 comprising the amino acid sequence of SEQ ID NO: 32 or a sequence differing in 1 or 2 amino acids therefrom;
(e)(i) a VH comprising complementarity-determining regions: CDR-H1 comprising the amino acid sequence of SEQ ID NO: 34 or a sequence differing in 1 or 2 amino acids therefrom, CDR-H2 comprising the amino acid sequence of SEQ ID NO: 35 or a sequence differing in 1 or 2 amino acids therefrom, and CDR-H3 comprising the amino acid sequence of SEQ ID NO: 36 or a sequence differing in 1 or 2 amino acids therefrom; and
   (ii) a VL comprising complementarity-determining regions: CDR-L1 comprising the amino acid sequence of SEQ ID NO: 38 or a sequence differing in 1 or 2 amino acids therefrom, CDR-L2 comprising the amino acid sequence of SEQ ID NO: 39 or a sequence differing in 1 or 2 amino acids therefrom, and CDR-L3 comprising the amino acid sequence of SEQ ID NO: 40 or a sequence differing in 1 or 2 amino acids therefrom;
(f)(i) a VH comprising complementarity-determining regions: CDR-H1 comprising the amino acid sequence of SEQ ID NO: 42 or a sequence differing in 1 or 2 amino acids therefrom, CDR-H2 comprising the amino acid sequence of SEQ ID NO: 43 or a sequence differing in 1 or 2 amino acids therefrom, and CDR-H3 comprising the amino acid sequence of SEQ ID NO: 44 or a sequence differing in 1 or 2 amino acids therefrom; and
   (ii) a VL comprising complementarity-determining regions:CDR-L1 comprising the amino acid sequence of SEQ ID NO: 46 or a sequence differing in 1 or 2 amino acids therefrom, CDR-L2 comprising the amino acid sequence of SEQ ID NO: 47 or a sequence differing in 1 or 2 amino acids therefrom, and CDR-L3 comprising the amino acid sequence of SEQ ID NO: 48 or a sequence differing in 1 or 2 amino acids therefrom;
(g)(i) a VH comprising complementarity-determining regions: CDR-H1 comprising the amino acid sequence of SEQ ID NO: 50 or a sequence differing in 1 or 2 amino acids therefrom, CDR-H2 comprising the amino acid sequence of SEQ ID NO: 51 or a sequence differing in 1 or 2 amino acids therefrom, and CDR-H3 comprising the amino acid sequence of SEQ ID NO: 52 or a sequence differing in 1 or 2 amino acids therefrom; and
   (ii) a VL comprising complementarity-determining regions: CDR-L1 comprising the amino acid sequence of SEQ ID NO: 54 or a sequence differing in 1 or 2 amino acids therefrom, CDR-L2 comprising the amino acid sequence of SEQ ID NO: 55 or a sequence differing in 1 or 2 amino acids therefrom, and CDR-L3 comprising the amino acid sequence of SEQ ID NO: 56 or a sequence differing in 1 or 2 amino acids therefrom;
(h)(i) a VH comprising complementarity-determining regions: CDR-H1 comprising the amino acid sequence of SEQ ID NO: 58 or a sequence differing in 1 or 2 amino acids therefrom, CDR-H2 comprising the amino acid sequence of SEQ ID NO: 59 or a sequence differing in 1 or 2 amino acids therefrom, and CDR-H3 comprising the amino acid sequence of SEQ ID NO: 60 or a sequence differing in 1 or 2 amino acids therefrom; and
   (ii) a VL comprising complementarity-determining regions: CDR-L1 comprising the amino acid sequence of SEQ ID NO: 62 or a sequence differing in 1 or 2 amino acids therefrom, CDR-L2 comprising the amino acid sequence of SEQ ID NO: 63 or a sequence differing in 1 or 2 amino acids therefrom, and CDR-L3 comprising the amino acid sequence of SEQ ID NO: 64 or a sequence differing in 1 or 2 amino acids therefrom;
(i)(i) a VH comprising complementarity-determining regions: CDR-H1 comprising the amino acid sequence of SEQ ID NO: 66 or a sequence differing in 1 or 2 amino acids therefrom, CDR-H2 comprising the amino acid sequence of SEQ ID NO: 67 or a sequence differing in 1 or 2 amino acids therefrom, and CDR-H3 comprising the amino acid sequence of SEQ ID NO: 68 or a sequence differing in 1 or 2 amino acids therefrom; and
   (ii) a VL comprising complementarity-determining regions: CDR-L1 comprising the amino acid sequence of SEQ ID NO: 70 or a sequence differing in 1 or 2 amino acids therefrom, CDR-L2 comprising the amino acid sequence of SEQ ID NO: 71 or a sequence differing in 1 or 2 amino acids therefrom, and CDR-L3 comprising the amino acid sequence of SEQ ID NO: 72 or a sequence differing in 1 or 2 amino acids therefrom;
(j)(i) a VH comprising complementarity-determining regions: CDR-H1 comprising the amino acid sequence of SEQ ID NO: 74 or a sequence differing in 1 or 2 amino acids therefrom, CDR-H2 comprising the amino acid sequence of SEQ ID NO: 75 or a sequence differing in 1 or 2 amino acids therefrom, and CDR-H3 comprising the amino acid sequence of SEQ ID NO: 76 or a sequence differing in 1 or 2 amino acids therefrom; and
   (ii) a VL comprising complementarity-determining regions: CDR-L1 comprising the amino acid sequence of SEQ ID NO: 78 or a sequence differing in 1 or 2 amino acids therefrom, CDR-L2 comprising the amino acid sequence of SEQ ID NO: 79 or a sequence differing in 1 or 2 amino acids therefrom, and CDR-L3 comprising the amino acid sequence of SEQ ID NO: 80 or a sequence differing in 1 or 2 amino acids therefrom;
(k)(i) a VH comprising complementarity-determining regions: CDR-H1 comprising the amino acid sequence of SEQ ID NO: 82 or a sequence differing in 1 or 2 amino acids therefrom, CDR-H2 comprising the amino acid sequence of SEQ ID NO: 83 or a sequence differing in 1 or 2 amino acids therefrom, and CDR-H3 comprising the amino acid sequence of SEQ ID NO: 84 or a sequence differing in 1 or 2 amino acids therefrom; and
   (ii) a VL comprising complementarity-determining regions: CDR-L1 comprising the amino acid sequence of SEQ ID NO: 86 or a sequence differing in 1 or 2 amino acids therefrom, CDR-L2 comprising the amino acid sequence of SEQ ID NO: 87 or a sequence differing in 1 or 2 amino acids therefrom, and CDR-L3 comprising the amino acid sequence of SEQ ID NO: 88 or a sequence differing in 1 or 2 amino acids therefrom;
(l)(i) a VH comprising complementarity-determining regions: CDR-H1 comprising the amino acid sequence of SEQ ID NO: 90 or a sequence differing in 1 or 2 amino acids therefrom, CDR-H2 comprising the amino acid sequence of SEQ ID NO: 91 or a sequence differing in 1 or 2 amino acids therefrom, and CDR-H3 comprising the amino acid sequence of SEQ ID NO: 92 or a sequence differing in 1 or 2 amino acids therefrom; and
   (ii) a VL comprising complementarity-determining regions: CDR-L1 comprising the amino acid sequence of SEQ ID NO: 94 or a sequence differing in 1 or 2 amino acids therefrom, CDR-L2 comprising the amino acid sequence of SEQ ID NO: 95 or a sequence differing in 1 or 2 amino acids therefrom, and CDR-L3 comprising the amino acid sequence of SEQ ID NO: 96 or a sequence differing in 1 or 2 amino acids therefrom;
(m)(i) a VH comprising complementarity-determining regions: CDR-H1 comprising the amino acid sequence of SEQ ID NO: 98 or a sequence differing in 1 or 2 amino acids therefrom, CDR-H2 comprising the amino acid sequence of SEQ ID NO: 99 or a sequence differing in 1 or 2 amino acids therefrom, and CDR-H3 comprising the amino acid sequence of SEQ ID NO: 100 or a sequence differing in 1 or 2 amino acids therefrom; and
   (ii) a VL comprising complementarity-determining regions: CDR-L1 comprising the amino acid sequence of SEQ ID NO: 102 or a sequence differing in 1 or 2 amino acids therefrom, CDR-L2 comprising the amino acid sequence of SEQ ID NO: 103 or a sequence differing in 1 or 2 amino acids therefrom, and CDR-L3 comprising the amino acid sequence of SEQ ID NO: 104 or a sequence differing in 1 or 2 amino acids therefrom; or
(n)(i) a VH comprising complementarity-determining regions: CDR-H1 comprising the amino acid sequence of SEQ ID NO: 106 or a sequence differing in 1 or 2 amino acids therefrom, CDR-H2 comprising the amino acid sequence of SEQ ID NO: 107 or a sequence differing in 1 or 2 amino acids therefrom, and CDR-H3 comprising the amino acid sequence of SEQ ID NO: 108 or a sequence differing in 1 or 2 amino acids therefrom; and
   (ii) a VL comprising complementarity-determining regions: CDR-L1 comprising the amino acid sequence of SEQ ID NO: 110 or a sequence differing in 1 or 2 amino acids therefrom, CDR-L2 comprising the amino acid sequence of SEQ ID NO: 111 or a sequence differing in 1 or 2 amino acids therefrom, and CDR-L3 comprising the amino acid sequence of SEQ ID NO: 112 or a sequence differing in 1 or 2 amino acids therefrom.

In some embodiments, the CDR-H1, CDR-H2, CDR-H3, CDR-L1, CDR-L2, and CDR-L3 comprise amino acid sequences that collectively differ by no more than two amino acid residues from the sequences of:
(a) SEQ ID NO: 2, SEQ ID NO: 3, SEQ ID NO: 4, SEQ ID NO: 6, SEQ ID NO: 7, and SEQ ID NO: 8, respectively;
(b) SEQ ID NO: 10, SEQ ID NO: 11, SEQ ID NO: 12, SEQ ID NO: 14, SEQ ID NO:15, and SEQ ID NO: 16, respectively;
(c) SEQ ID NO: 18, SEQ ID NO: 19, SEQ ID NO: 20, SEQ ID NO: 22, SEQ ID NO:23, SEQ ID NO: 24, respectively;
(d) SEQ ID NO: 26, SEQ ID NO: 27, SEQ ID NO: 28, SEQ ID NO: 30, SEQ ID NO: 31, SEQ ID NO: 32, respectively;
(e) SEQ ID NO: 34, SEQ ID NO: 35, SEQ ID NO: 36, SEQ ID NO: 38, SEQ ID NO: 39, SEQ ID NO: 40, respectively;
(f) SEQ ID NO: 42, SEQ ID NO: 43, SEQ ID NO: 44, SEQ ID NO: 46, SEQ ID NO: 47, SEQ ID NO: 48, respectively;
(g) SEQ ID NO: 50, SEQ ID NO: 51, SEQ ID NO: 52, SEQ ID NO: 54, SEQ ID NO: 55, SEQ ID NO: 56, respectively;
(h) SEQ ID NO: 58, SEQ ID NO: 59, SEQ ID NO: 60, SEQ ID NO: 62, SEQ ID NO: 63, SEQ ID NO: 64, respectively;
(i) SEQ ID NO: 66, SEQ ID NO: 67, SEQ ID NO: 68, SEQ ID NO: 70, SEQ ID NO:71, SEQ ID NO: 72, respectively;
(j) SEQ ID NO: 74, SEQ ID NO: 75, SEQ ID NO: 76, SEQ ID NO: 78, SEQ ID NO: 79, SEQ ID NO: 80, respectively;
(k) SEQ ID NO: 82, SEQ ID NO: 83, SEQ ID NO: 84, SEQ ID NO: 86, SEQ ID NO: 87, SEQ ID NO: 88, respectively;
(l) SEQ ID NO: 90, SEQ ID NO: 91, SEQ ID NO: 92, SEQ ID NO: 94, SEQ ID NO: 95, SEQ ID NO: 96, respectively;
(m) SEQ ID NO: 98, SEQ ID NO: 99, SEQ ID NO: 100, SEQ ID NO: 102, SEQ ID NO: 103, SEQ ID NO: 104, respectively; or
(n) SEQ ID NO: 106, SEQ ID NO: 107, SEQ ID NO: 108, SEQ ID NO: 110, SEQ ID NO: 111, SEQ ID NO: 112, respectively.

In some embodiments, said antibody or antigen-binding fragment thereof comprises:
(a)(i) a VH comprising complementarity-determining regions: CDR-H1 comprising the amino acid sequence of SEQ ID NO: 2, CDR-H2 comprising the amino acid sequence of SEQ ID NO: 3, and CDR-H3 comprising the amino acid sequence of SEQ ID NO: 4; and
   (ii) a VL comprising complementarity-determining regions: CDR-L1 comprising the amino acid sequence of SEQ ID NO: 6, CDR-L2 comprising the amino acid sequence of SEQ ID NO: 7, and CDR-L3 comprising the amino acid sequence of SEQ ID NO: 8;
(b)(i) a VH comprising complementarity-determining regions: CDR-H1 comprising the amino acid sequence of SEQ ID NO: 10, CDR-H2 comprising the amino acid sequence of SEQ ID NO: 11, and CDR-H3 comprising the amino acid sequence of SEQ ID NO: 12; and
   (ii) a VL comprising complementarity-determining regions: CDR-L1 comprising the amino acid sequence of SEQ ID NO: 14, CDR-L2 comprising the amino acid sequence of SEQ ID NO: 15, and CDR-L3 comprising the amino acid sequence of SEQ ID NO: 16;
(c)(i) a VH comprising complementarity-determining regions: CDR-H1 comprising the amino acid sequence of SEQ ID NO: 18, CDR-H2 comprising the amino acid sequence of SEQ ID NO: 19, and CDR-H3 comprising the amino acid sequence of SEQ ID NO: 20; and
   (ii) a VL comprising complementarity-determining regions: CDR-L1 comprising the amino acid sequence of SEQ ID NO: 22, CDR-L2 comprising the amino acid sequence of SEQ ID NO: 23, and CDR-L3 comprising the amino acid sequence of SEQ ID NO: 24;
(d)(i) a VH comprising complementarity-determining regions: CDR-H1 comprising the amino acid sequence of SEQ ID NO: 26, CDR-H2 comprising the amino acid sequence of SEQ ID NO: 27, and CDR-H3 comprising the amino acid sequence of SEQ ID NO: 28; and
   (ii) a VL comprising complementarity-determining regions: CDR-L1 comprising the amino acid sequence of SEQ ID NO: 30, CDR-L2 comprising the amino acid sequence of SEQ ID NO: 31, and CDR-L3 comprising the amino acid sequence of SEQ ID NO: 32;
(e)(i) a VH comprising complementarity-determining regions: CDR-H1 comprising the amino acid sequence of SEQ ID NO: 34, CDR-H2 comprising the amino acid sequence of SEQ ID NO: 35, and CDR-H3 comprising the amino acid sequence of SEQ ID NO: 36; and
   (ii) a VL comprising complementarity-determining regions: CDR-L1 comprising the amino acid sequence of SEQ ID NO: 38, CDR-L2 comprising the amino acid sequence of SEQ ID NO: 39, and CDR-L3 comprising the amino acid sequence of SEQ ID NO: 40;
(f)(i) a VH comprising complementarity-determining regions: CDR-H1 comprising the amino acid sequence of SEQ ID NO: 42, CDR-H2 comprising the amino acid sequence of SEQ ID NO: 43, and CDR-H3 comprising the amino acid sequence of SEQ ID NO: 44; and
   (ii) a VL comprising complementarity-determining regions: CDR-L1 comprising the amino acid sequence of SEQ ID NO: 46, CDR-L2 comprising the amino acid sequence of SEQ ID NO: 47, and CDR-L3 comprising the amino acid sequence of SEQ ID NO: 48;
(g)(i) a VH comprising complementarity-determining regions: CDR-H1 comprising the amino acid sequence of SEQ ID NO: 50, CDR-H2 comprising the amino acid sequence of SEQ ID NO: 51, and CDR-H3 comprising the amino acid sequence of SEQ ID NO: 52; and
   (ii) a VL comprising complementarity-determining regions: CDR-L1 comprising the amino acid sequence of SEQ ID NO: 54, CDR-L2 comprising the amino acid sequence of SEQ ID NO: 55, and CDR-L3 comprising the amino acid sequence of SEQ ID NO: 56;
(h)(i) a VH comprising complementarity-determining regions: CDR-H1 comprising the amino acid sequence of SEQ ID NO: 58, CDR-H2 comprising the amino acid sequence of SEQ ID NO: 59, and CDR-H3 comprising the amino acid sequence of SEQ ID NO: 60; and
   (ii) a VL comprising complementarity-determining regions: CDR-L1 comprising the amino acid sequence of SEQ ID NO: 62, CDR-L2 comprising the amino acid sequence of SEQ ID NO: 63, and CDR-L3 comprising the amino acid sequence of SEQ ID NO: 64;
(i)(i) a VH comprising complementarity-determining regions: CDR-H1 comprising the amino acid sequence of SEQ ID NO: 66, CDR-H2 comprising the amino acid sequence of SEQ ID NO: 67, and CDR-H3 comprising the amino acid sequence of SEQ ID NO: 68; and
   (ii) a VL comprising complementarity-determining regions: CDR-L1 comprising the amino acid sequence of SEQ ID NO: 70, CDR-L2 comprising the amino acid sequence of SEQ ID NO: 71, and CDR-L3 comprising the amino acid sequence of SEQ ID NO: 72;
(j)(i) a VH comprising complementarity-determining regions: CDR-H1 comprising the amino acid sequence of SEQ ID NO: 74, CDR-H2 comprising the amino acid sequence of SEQ ID NO: 75, and CDR-H3 comprising the amino acid sequence of SEQ ID NO: 76; and
   (ii) a VL comprising complementarity-determining regions: CDR-L1 comprising the amino acid sequence of SEQ ID NO: 78, CDR-L2 comprising the amino acid sequence of SEQ ID NO: 79, and CDR-L3 comprising the amino acid sequence of SEQ ID NO: 80;
(k)(i) a VH comprising complementarity-determining regions: CDR-H1 comprising the amino acid sequence of SEQ ID NO: 82, CDR-H2 comprising the amino acid sequence of SEQ ID NO: 83, and CDR-H3 comprising the amino acid sequence of SEQ ID NO: 84; and
   (ii) a VL comprising complementarity-determining regions: CDR-L1 comprising the amino acid sequence of SEQ ID NO: 86, CDR-L2 comprising the amino acid sequence of SEQ ID NO: 87, and CDR-L3 comprising the amino acid sequence of SEQ ID NO: 88;
(l)(i) a VH comprising complementarity-determining regions: CDR-H1 comprising the amino acid sequence of SEQ ID NO: 90, CDR-H2 comprising the amino acid sequence of SEQ ID NO: 91, and CDR-H3 comprising the amino acid sequence of SEQ ID NO: 92; and
   (ii) a VL comprising complementarity-determining regions: CDR-L1 comprising the amino acid sequence of SEQ ID NO: 94, CDR-L2 comprising the amino acid sequence of SEQ ID NO: 95, and CDR-L3 comprising the amino acid sequence of SEQ ID NO: 96;
(m)(i) a VH comprising complementarity-determining regions: CDR-H1 comprising the amino acid sequence of SEQ ID NO: 98, CDR-H2 comprising the amino acid sequence of SEQ ID NO: 99, and CDR-H3 comprising the amino acid sequence of SEQ ID NO: 100; and
   (ii) a VL comprising complementarity-determining regions: CDR-L1 comprising the amino acid sequence of SEQ ID NO: 102, CDR-L2 comprising the amino acid sequence of SEQ ID NO: 103, and CDR-L3 comprising the amino acid sequence of SEQ ID NO: 104; or
(n)(i) a VH comprising complementarity-determining regions: CDR-H1 comprising the amino acid sequence of SEQ ID NO: 106, CDR-H2 comprising the amino acid sequence of SEQ ID NO: 107, and CDR-H3 comprising the amino acid sequence of SEQ ID NO: 108; and
   (ii) a VL comprising complementarity-determining regions: CDR-L1 comprising the amino acid sequence of SEQ ID NO: 110, CDR-L2 comprising the amino acid sequence of SEQ ID NO: 111, and CDR-L3 comprising the amino acid sequence of SEQ ID NO: 112.

In some embodiments, the antibody or antigen binding fragment thereof comprises:
(i) a VH comprising complementarity-determining regions: CDR-H1 comprising the amino acid sequence of SEQ ID NO: 2; CDR-H2 comprising the amino acid sequence of SEQ ID NO: 3; and CDR-H3 comprising the amino acid sequence of SEQ ID NO: 4; and
(ii) a VL comprising complementarity-determining regions: CDR-L1 comprising the amino acid sequence of SEQ ID NO: 6; CDR-L2 comprising the amino acid sequence of SEQ ID NO: 7; and CDR-L3 comprising the amino acid sequence of SEQ ID NO: 8.

In some embodiments, the antibody or antigen binding fragment thereof comprises: a VH comprises an amino acid sequence that is at least 85% identical to that of SEQ ID NO: 1; and a VL comprises an amino acid sequence that is at least 85% identical to that of SEQ ID NO: 5.

In some embodiments, the antibody or antigen binding fragment thereof comprises: a VH comprising the amino acid sequence of SEQ ID NO: 1; and a VL comprising the amino acid sequence of SEQ ID NO: 5.

In some embodiments, the antibody or antigen binding fragment thereof comprises:
(i) a VH comprising complementarity-determining regions: CDR-H1 comprising the amino acid sequence of SEQ ID NO: 82; CDR-H2 comprising the amino acid sequence of SEQ ID NO: 83; and CDR-H3 comprising the amino acid sequence of SEQ ID NO: 84; and
(ii) a VL comprising complementarity-determining regions: CDR-L1 comprising the amino acid sequence of SEQ ID NO: 86; CDR-L2 comprising the amino acid sequence of SEQ ID NO: 87; and CDR-L3 comprising the amino acid sequence of SEQ ID NO: 88.

In some embodiments, the antibody or antigen binding fragment thereof comprises: a VH comprises an amino acid sequence that is at least 85% identical to that of SEQ ID NO: 81; and a VL comprises an amino acid sequence that is at least 85% identical to that of SEQ ID NO: 85.

In some embodiments, the antibody or antigen binding fragment thereof comprises: a VH comprising the amino acid sequence of SEQ ID NO: 81; and a VL comprising the amino acid sequence of SEQ ID NO: 85.

In some embodiments, the antibody or antigen binding fragment thereof comprises:
(a) the VH comprises an amino acid sequence at least 90%, 95%, 96%, 97%, 98%, or 99% identical to SEQ ID NO: 1 and the VL comprises an amino acid sequence at least 90%, 95%, 96%, 97%, 98%, or 99% identical to SEQ ID NO: 5;
(b) the VH comprises an amino acid sequence at least 90%, 95%, 96%, 97%, 98%, or 99% identical to SEQ ID NO: 9 and the VL comprises an amino acid sequence at least 90%, 95%, 96%, 97%, 98%, or 99% identical to SEQ ID NO: 13;
(c) the VH comprises an amino acid sequence at least 90%, 95%, 96%, 97%, 98%, or 99% identical to SEQ ID NO: 17 and the VL comprises an amino acid sequence at least 90%, 95%, 96%, 97%, 98%, or 99% identical to SEQ ID NO: 21;
(d) the VH comprises an amino acid sequence at least 90%, 95%, 96%, 97%, 98%, or 99% identical to SEQ ID NO: 25 and the VL comprises an amino acid sequence at least 90%, 95%, 96%, 97%, 98%, or 99% identical to SEQ ID NO: 29;
(e) the VH comprises an amino acid sequence at least 90%, 95%, 96%, 97%, 98%, or 99% identical to SEQ ID NO: 33 and the VL comprises an amino acid sequence at least 90%, 95%, 96%, 97%, 98%, or 99% identical to SEQ ID NO: 37;
(f) the VH comprises an amino acid sequence at least 90%, 95%, 96%, 97%, 98%, or 99% identical to SEQ ID NO: 41 and the VL comprises an amino acid sequence at least 90%, 95%, 96%, 97%, 98%, or 99% identical to SEQ ID NO: 45;
(g) the VH comprises an amino acid sequence at least 90%, 95%, 96%, 97%, 98%, or 99% identical to SEQ ID NO: 49 and the VL comprises an amino acid sequence at least 90%, 95%, 96%, 97%, 98%, or 99% identical to SEQ ID NO: 53;
(h) the VH comprises an amino acid sequence at least 90%, 95%, 96%, 97%, 98%, or 99% identical to SEQ ID NO: 57 and the VL comprises an amino acid sequence at least 90%, 95%, 96%, 97%, 98%, or 99% identical to SEQ ID NO: 61;
(i) the VH comprises an amino acid sequence at least 90%, 95%, 96%, 97%, 98%, or 99% identical to SEQ ID NO: 65 and the VL comprises an amino acid sequence at least 90%, 95%, 96%, 97%, 98%, or 99% identical to SEQ ID NO: 69;
(j) the VH comprises an amino acid sequence at least 90%, 95%, 96%, 97%, 98%, or 99% identical to SEQ ID NO: 73 and the VL comprises an amino acid sequence at least 90%, 95%, 96%, 97%, 98%, or 99% identical to SEQ ID NO: 77;
(k) the VH comprises an amino acid sequence at least 90%, 95%, 96%, 97%, 98%, or 99% identical to SEQ ID NO: 81 and the VL comprises an amino acid sequence at least 90%, 95%, 96%, 97%, 98%, or 99% identical to SEQ ID NO: 85;
(l) the VH comprises an amino acid sequence at least 90%, 95%, 96%, 97%, 98%, or 99% identical to SEQ ID NO: 89 and the VL comprises an amino acid sequence at least 90%, 95%, 96%, 97%, 98%, or 99% identical to SEQ ID NO: 93;
(m) the VH comprises an amino acid sequence at least 90%, 95%, 96%, 97%, 98%, or 99% identical to SEQ ID NO: 97 and the VL comprises an amino acid sequence at least 90%, 95%, 96%, 97%, 98%, or 99% identical to SEQ ID NO: 101; or
(n) the VH comprises an amino acid sequence at least 90%, 95%, 96%, 97%, 98%, or 99% identical to SEQ ID NO: 105 and the VL comprises an amino acid sequence at least 90%, 95%, 96%, 97%, 98%, or 99% identical to SEQ ID NO: 109.

In some embodiments, the antibody or antigen binding fragment thereof comprises: (a) the VH comprises an amino acid sequence of SEQ ID NO: 1 and the VL comprises an amino acid sequence of SEQ ID NO: 5; (b) the VH comprises an amino acid sequence of SEQ ID NO: 9 and the VL comprises an amino acid sequence of SEQ ID NO: 13; (c) the VH comprises an amino acid sequence of SEQ ID NO: 17 and the VL comprises an amino acid sequence of SEQ ID NO: 21; (d) the VH comprises an amino acid sequence of SEQ ID NO: 25 and the VL comprises an amino acid sequence of SEQ ID NO: 29; (e) the VH comprises an amino acid sequence of SEQ ID NO: 33 and the VL comprises an amino acid sequence of SEQ ID NO: 37; (f) the VH comprises an amino acid sequence of SEQ ID NO: 41 and the VL comprises an amino acid sequence of SEQ ID NO: 45; (g) the VH comprises an amino acid sequence of SEQ ID NO: 49 and the VL comprises an amino acid sequence of SEQ ID NO: 53; (h) the VH comprises an amino acid sequence of SEQ ID NO: 57 and the VL comprises an amino acid sequence of SEQ ID NO: 61; (i) the VH comprises an amino acid sequence of SEQ ID NO: 65 and the VL comprises an amino acid sequence of SEQ ID NO: 69; (j) the VH comprises an amino acid sequence of SEQ ID NO: 73 and the VL comprises an amino acid sequence of SEQ ID NO: 77; (k) the VH comprises an amino acid sequence of SEQ ID NO: 81 and the VL comprises an amino acid sequence of SEQ ID NO: 85; (1) the VH comprises an amino acid sequence of SEQ ID NO: 89 and the VL comprises an amino acid sequence of SEQ ID NO: 93; (m) the VH comprises an amino acid sequence of SEQ ID NO: 97 and the VL comprises an amino acid sequence of SEQ ID NO: 101; or (n) the VH comprises an amino acid sequence of SEQ ID NO: 105 and the VL comprises an amino acid sequence of SEQ ID NO: 109.

In some embodiments, the antibody or antigen binding fragment thereof is capable of binding to mouse CLDN18.2. In some embodiments, the antibody or antigen binding fragment thereof is not capable of significantly binding to CLDN18.1.

In some embodiments, the antibody is a human antibody. In some embodiments, the antigen binding fragment is a Fab, a F(ab′)₂, a Fab′, a single-chain Fv (scFv), an Fv fragment, a Fd fragment, a diabody, or an isolated CDR or a combination of two or more isolated CDRs optionally joined by a linker. In some embodiments, the antigen binding fragment is an scFv.

In some embodiments, the antibody or antigen binding fragment thereof comprises an antibody heavy chain constant region. In some embodiments, the heavy chain constant region is a human IgG heavy chain constant region. In some embodiments, the antibody heavy chain constant region is a human IgG4 heavy chain constant region. In some embodiments, the human IgG4 heavy chain constant region comprises a S228P mutation.

In some embodiments, the compound is capable of binding to human CLDN18.2 with a K_{d} value of at most about 15 nM, at most about 12.5 nM, at most about 10 nM, at most about 7.5 nM, at most about 5 nM, or at most about 2.5.

In some embodiments, the compound comprises the following structure:

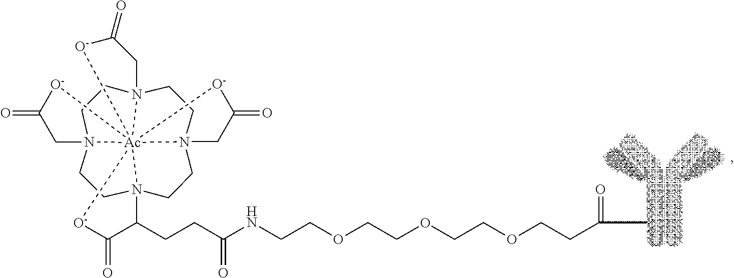

wherein [symbol] is an antibody or an antigen-binding fragment thereof that specifically binds to CLDN 18.2 or a fragment thereof. In some embodiments, the antibody or an antigen-binding fragment thereof is linked to A-L- via the side-chain amino group of a lysine residue

In another aspect, the present invention also relates to a pharmaceutical composition comprising one of the compounds described above and a pharmaceutically acceptable carrier, diluent, or excipient.

Still within the scope of this invention is a method of radiation treatment planning and/or radiation treatment and the method comprising administering to a subject in need thereof one of the compounds set forth above or a pharmaceutical composition comprising the same.

Further covered by this invention is a method of treating cancer comprising cells expressing CLDN 18.2, the method comprising administering to a subject (e.g., a human) in need thereof a first dose of a compound or composition provided above in an amount effective for radiation treatment planning, followed by administering subsequent doses of a compound or composition provided above in a therapeutically effective amount.

In some embodiments, the compound or composition administered in the first dose and the compound or composition administered a subsequent dose are the same.

In some embodiments, the compound or composition administered in the first dose and the compound or composition administered a subsequent are different.

In some embodiments, the cancer comprising cells expressing CLDN 18.2 is esophageal cancer (e.g., esophageal adenocarcinoma), gastric cancer, or pancreatic cancer.

In some embodiments, the method of treatment of this invention further comprises administering to a subject (e.g., a human) in need thereof an antiproliferative agent, radiation sensitizer, an immunoregulatory or immunomodulatory agent.

### BRIEF DESCRIPTION OF THE DRAWINGS

FIG. 1A is a schematic depicting the general structure of bifunctional chelate comprising a chelate, a linker, and a cross-linking group.

FIG. 1B is a schematic depicting the general structure of a bifunctional conjugate comprising a chelate, a linker, and a targeting moiety.

FIG. 1C and FIG. 1D are schematics depicting the structures of [¹⁷⁷Lu]-DOTA-anti-CLDN 18.2 and [²²⁵Ac]-DOTA-anti-CLDN 18.2, two exemplary CLDN 18.2 radioimmunoconjugates disclosed herein.

FIG. 2 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in Example 2.

FIG. 3 is a schematic depicting the synthesis of the bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in Example 3.

FIG. 4 is a schematic depicting the conjugation and radiolabeling for synthesis of [¹⁷⁷Lu]-Compound C-anti-CLDN 18.2 conjugates. See Example 4.

FIG. 5A shows binding curves for [¹⁷⁷Lu]-Compound C-anti-CLDN 18.2 conjugates, i.e., Compounds F and G, binding to MiaPaCa2 K18.2 cells. FIG. 5B shows binding curves for Compound F binding to PATU8988S cells. See Example 5.

FIG. 6 shows the results of internalization (or in vitro residualization) of Compound F in MiaPaCa2 K18.2 and PATU8988S cells. See Example 6.

FIG. 7 shows a plot representing the results of biodistribution studies in a MiaPaCa2 K18.2 model and injected with Compound F. Percentage injected dose per gram of tissue (% ID/g) is plotted on the x-axis and is shown for blood, heart, intestines, esophagus, pancreas, stomach, kidneys, liver, lungs, spleen, tumor, urine, and tail at 4, 24, 96, and 168 hours. See Example 7.

FIG. 8 shows a plot representing the results of biodistribution studies in a MiaPaCa2 K18.2 model and injected with Compound G. Percentage injected dose per gram of tissue (% ID/g) is plotted on the x-axis and is shown for blood, heart, intestines, esophagus, pancreas, stomach, kidneys, liver, lungs, spleen, tumor, urine, and tail at 4, 24, 96, and 168 hours. See Example 7.

FIG. 9 shows results of in vivo efficacy studies in a MiaPaCa2 K18.2 model and injected with [²²⁵Ac]-Compound C-anti-CLDN 18.2 at various doses. See Example 8.

### DETAILED DESCRIPTION

Radioimmunoconjugates are designed to target a protein or receptor that is upregulated in a disease state to deliver a radioactive payload to damage and kill cells of interest (radioimmunotherapy). The process of delivering such a payload, via radioactive decay, produces an alpha, beta, gamma particle or Auger electron that can cause direct effects to DNA (such as single or double stranded DNA breaks) or indirect effects such as by-stander or crossfire effects.

Radioimmunoconjugates typically contain a biological targeting moiety (e.g., an antibody or antigen binding fragment thereof that is capable of specifically binding to human Claudin 18.2), a radioisotope, and a molecule that links the two. Conjugates are formed when a bifunctional chelate is appended to the biological targeting molecule so that structural alterations are minimal while maintaining target affinity. Once radiolabelled, the final radioimmunoconjugate is formed.

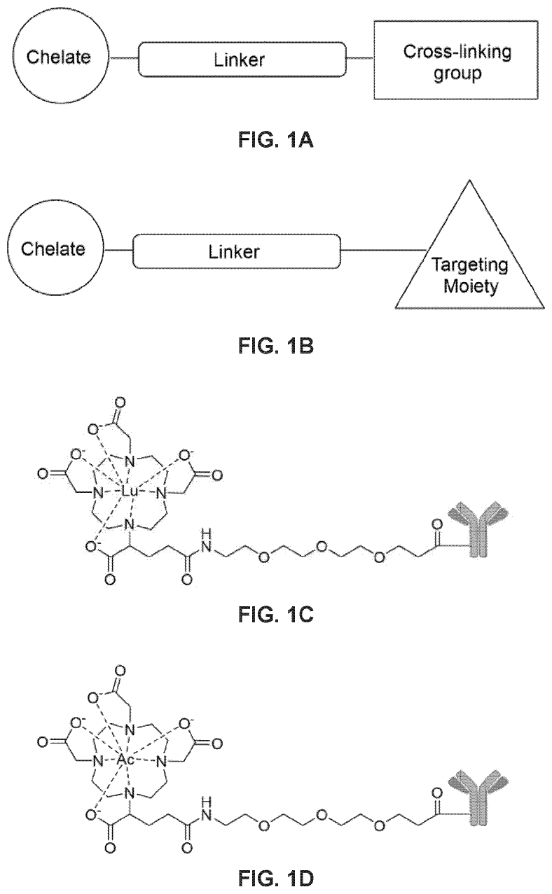

Bifunctional chelates structurally contain a chelate, a linker, and a cross-linking group (FIG. 1A). When developing new bifunctional chelates, most efforts focus on the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required by an antibody drug conjugate (“ADC”), in order to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new therapeutic radioimmunoconjugates.

One potential method for reducing off-target exposure is to remove the radioactivity more effectively from the body (e.g., from normal tissue in the body). One mechanism is to increase the rate of clearance of the biological targeting agent. This approach likely requires identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional chelate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12(11), 2472-2482; Methods Mol Biol. 2009, 539, 191-211; Bioconjug Chem. 2003, 14(5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, compounds, e.g., radioimmunoconjugates, that are more effectively eliminated from the body after catabolism and/or metabolism, thereby more effectively eliminating radioactivity from the body while maintaining therapeutic efficacy. This unexpected superiority is achieved by making modifications to the linker region of the bifunctional chelate.

Disclosed immunoconjugates may, in some embodiments, achieve a reduction of total body radioactivity, for example, by increasing the extent of excretion of the catabolic/metabolic products while maintaining the pharmacokinetics of the intact molecule when compared to known bifunctional chelates. In some embodiments, this reduction in radioactivity results from the clearance of catabolic/metabolic by-products without impacting other in vitro and in vivo properties such as binding specificity (in vitro binding), cellular retention, and tumor uptake in vivo. Thus, in some embodiments, provided compounds achieve reduced radioactivity in the human body while maintaining on-target activity.

### Definitions

As used herein, the term “bind” or “binding” of a targeting moiety means an at least temporary interaction or association with or to a target molecule, e.g., human Claudin 18.2 (CLDN 18.2), as described herein.

The terms “bifunctional chelate,” as used herein, refers to a compound that comprises a chelate, a linker, and a cross-linking group. See, e.g., FIG. 1A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See, e.g., FIG. 1B.

The term “cancer,” as used herein, refers to any cancer caused by the proliferation of malignant neoplastic cells, such as tumors, neoplasms, carcinomas, sarcomas, leukemias, and lymphomas. In some embodiments, a cancer of the present disclosure comprises cells (e.g., tumor cells) expressing CLDN 18.2, such as, but not limited to, esophageal cancer (e.g., esophageal adenocarcinoma), gastric cancer, and pancreatic cancer.

The term “chelate,” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

As used herein, unless otherwise noted, the phrase “constant region,” when used in reference to an antibody or a fragment thereof (e.g., an IgG1, an IgG2, or an IgG4 constant region) is intended to encompass both wild type constant regions and variants (e.g., constant regions having at least 85%, at least 90%, at least 92%, at least 94%, at least 95%, at least 96%, at least 97%, at least 98%, or at least 99% amino acid sequence identity with a reference sequence for a wild-type constant region.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ³H, ¹⁴C, ¹⁵N, ¹⁸F, ³⁵S ⁴⁴Sc, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be used as therapeutic agents, e.g., an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 3, 4, 5, 6, 7, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. A radioimmunoconjugate provided in the present disclosure typically refers to a bifunctional conjugate that comprises a metal complex formed from a radioisotope or radionuclide.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a radioimmunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide,” as used herein, refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

As used herein, the term “targeting moiety” refers to any molecule or any part of a molecule that is capable of binding to a given target. The term, “Claudin 18.2 targeting moiety” refers to a targeting moiety that is capable of binding to a Claudin 18.2 molecule, e.g., a human Claudin 18.2.

As used herein, the term “fragment,” when used to refer to a Claudin 18.2 fragment, refers to N-terminally and/or C-terminally truncated Claudin 18.2 or protein domains of Claudin 18.2. Unless otherwise noted, fragments of Claudin 18.2 used in accordance with embodiments described herein retain the capability of the full-length Claudin 18.2 to be recognized and/or bound by an Claudin 18.2-targeting moiety as described in the present disclosure.

The use of the terms “a” and “an” and “the” and similar referents in the context of describing the disclosure (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the specificity of the binding characteristics of the parent antibody.

Antibodies or antigen-binding fragments thereof of the present disclosure may be isolated and/or substantially purified.

### Compounds, e.g., Immunoconjugates or Radioimmunoconjugates

In one aspect, this disclosure provides compounds, e.g., immunoconjugates or radioimmunoconjugates, comprising the following structure, or pharmaceutically acceptable salts thereof:

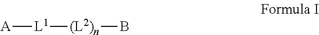

wherein
A is a chelating moiety or a metal complex thereof,
B is a targeting moiety that is capable of binding to Claudin 18.2 (CLDN18.2) or a fragment thereof;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

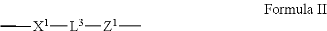

wherein
   X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
   L³ is optionally substituted C₁-C₅₀ alkyl or optionally substituted C₁-C₅₀ heteroalkyl; and
   Z¹ is —CH₂— #, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

Typical substituents of alkyl, heteroalkyl, aryl, or heteroaryl include, but are not limited to halo (e.g., F, Cl, Br, I), OH, CN, nitro, amino, C₁₋₆ alkyl, C₂₋₆ alkenyl, C₂₋₆ alkynyl, C₃₋₈ cycloalkyl, C₁₋₆ heteroalkyl, C₁₋₆ heterocycloalkyl, haloalkyl (e.g., CF₃), alkoxy (e.g., OCH₃), alkylamino (e.g., NH₂CH₃), sulfonyl, aryl, and heteroaryl.

In some embodiments, the compound (e.g., immunoconjugate or radioimmunoconjugate) has or comprises the structure shown below:

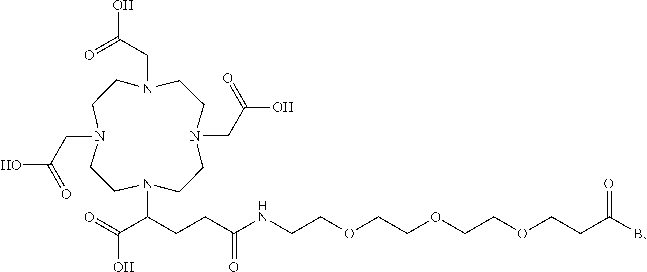

wherein B is an CLDN18.2 targeting moiety (e.g., a CLDN18.2 antibody or antigen-binding fragment thereof).

In some embodiments, A-L- comprises one of the following structures, or a metal complex thereof:

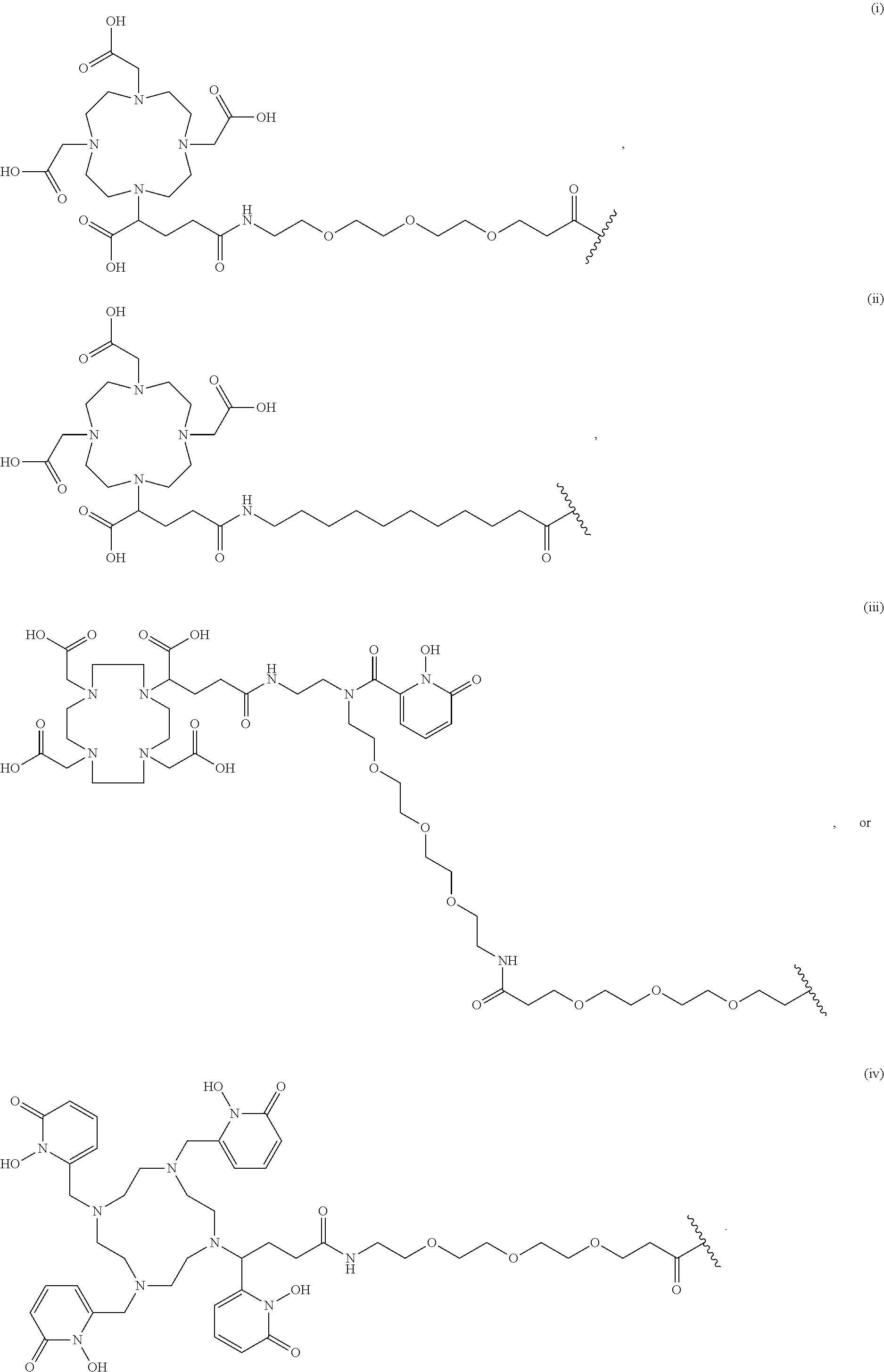 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O ; *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, provided compounds (e.g., immunoconjugates or radioimmunoconjugates) are capable of binding to human CLDN18.2 with a K_{d} value of at most about 15 nM, at most about 12.5 nM, at most about 10 nM, at most about 7.5 nM, at most about 7 nM, at most about 6.5 nM, at most about 6 nM, at most about 5 nM, at most about 4 nM, at most about 3.5 nM, at most about 3 nM, or at most about 2.5 nM. In some embodiments, provided compounds (e.g., immunoconjugates or radioimmunoconjugates) are capable of binding to human CLDN18.2 with a K_{d} value of about 15 nM, about 12.5 nM, about 10 nM, about 7.5 nM, about 7 nM, about 6.5 nM, about 6 nM, about 5 nM, about 4 mM, about 3.5 nM, about 3 nM, or about 2.5 nM. In some embodiments, provided compounds (e.g., immunoconjugates or radioimmunoconjugates) are capable of binding to human CLDN18.2 with a K_{d} value of at most about 6.75 nM. In some embodiments, provided compounds (e.g., immunoconjugates or radioimmunoconjugates) are capable of binding to human CLDN18.2 with a K_{d} value of at most about 2.75 nM.

In some embodiments, as further described herein, the compound (e.g., immunoconjugate or radioimmunoconjugate) comprises a chelating moiety or a metal complex thereof, which metal complex may comprise a radionuclide. In some such compounds, the average ratio or median ratio of the chelating moiety to the CLDN18.2 targeting moiety is eight or less, seven or less, six or less, five or less, four or less, three or less, two or less, or about one. In some compounds, the average ratio or median ratio of the chelating moiety to the CLDN18.2 targeting moiety is about one.

In some embodiments, after a radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a compound (e.g., immunoconjugate or radioimmunoconjugate) has been administered to a mammal, the compound (e.g., immunoconjugate or radioimmunoconjugate) exhibits decreased off-target binding effects (e.g., toxicities) as compared to a reference compound (e.g., reference conjugate, e.g., a reference immunoconjugate such as a reference radioimmunoconjugate). In some embodiments, this decreased off-target binding effect is a feature of a compound (e.g., immunoconjugate or radioimmunoconjugate) that also exhibits a greater excretion rate as described herein.

### Targeting Moieties

Targeting moieties include any molecule or any part of a molecule that is capable of binding (e.g., capable of specifically binding, specifically binds to, etc.) to a given target, e.g., CLDN18.2. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins). In some embodiments, a moiety is both a targeting and a therapeutic moiety, i.e., the moiety is capable of binding to a given target and also confers a therapeutic benefit. In some embodiments, the targeting moiety comprises a small molecule.

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa.

Typically, the targeting moiety is capable of binding to CLDN18.2, or a fragment thereof. In some embodiments, the targeting moiety is capable of binding to human CLDN18.2, or a fragment thereof. In some embodiments, the targeting moiety is capable of binding to mouse CLDN18.2, or a fragment thereof.

In some embodiments, the targeting moiety does not bind to Claudin 18.1 with detectable affinity. By “detectable affinity,” it is generally meant that the binding ability between a targeting moiety and its target, reported by a K_{D}, EC₅₀, or IC₅₀ value, is at most about 10⁵ M or lower. A binding affinity, reported by a K_{D}, EC₅₀, or IC₅₀ value, higher than 10⁵ M is generally no longer measurable with common methods such as ELISA and flow cytometry, and is, therefore, of secondary importance.

In some embodiments, the targeting moiety inhibits CLDN 18.2. By “inhibits,” it is meant that the targeting moiety at least partially inhibits one or more functions of CLDN 18.2 (e.g., human CLDN 18.2). In some embodiments, the targeting moiety impairs signaling downstream of CLDN 18.2, e.g., results in the suppressed growth of CLDN 18.2-positive tumor cells.

### Antibodies and Antigen-Binding Moieties

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies suitable for use with the present disclosure can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or antigen-binding fragments described herein can be produced by any method known in the art for the synthesis of antibodies (See, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991), or any of the molecules described herein.

“Avimer” relates to a multimeric binding protein or peptide engineered using, for example, in vitro exon shuffling and phage display. Multiple binding domains are linked, resulting in greater affinity and specificity compared to single epitope immunoglobin domains.

“Nanobodies” are antibody fragments consisting of a single monomeric variable antibody domain. Nanobodies may also be referred to as single-domain antibodies. Like antibodies, nanobodies are capable of binding selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display). “Affibodies” are polypeptides or proteins engineered to bind to a specific antigen. As such, affibodies may be considered to mimic certain functions of antibodies.

Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g. insulin, fibrinogen, transferrin, tumor necrosis factor-α, IL-8, gp120, CD28, human serum albumin, IgA, IgE, IgM, HER2 and EGFR) have been generated, demonstrating affinities (Kd) in the μM to pM range. “Diabodies” are antibody fragments with two antigen-binding sites that may be bivalent or bispecific. See for example Hudson et al., (2003). Single-chain antibodies are antibody fragments comprising all or a portion of the heavy chain variable domain or all, or a portion of the light chain variable domain of an antibody. Antibody fragments can be made by various techniques including but not limited to proteolytic digestion of an intact antibody as well as production by recombinant hosts (e.g., E. coli or phage) as described herein.

In certain embodiments, the antibody or antigen-binding fragment thereof is a multispecific, e.g. bispecific. Multispecific antibodies (or antigen-binding fragments thereof) include monoclonal antibodies (or antigen-binding fragments thereof) that have binding specificities for at least two different sites.

In some embodiments, provided herein are compounds (e.g., immunoconjugates or radioimmunoconjugates) comprising antibodies or antigen-binding fragments that comprise a heavy chain variable domain and a light chain variable domain, wherein the heavy chain variable domain comprises a CDR-H1, CDR-H2, and CDR-H3, and the light chain variable domain comprises a CDR-L1, CDR-L2, and CDR-L3, wherein the CDR-H1, CDR-H2, CDR-H3, CDR-L1, CDR-L2, and CDR-L3, are those of an antibody described in Table 1 below. In some embodiments, provided are compounds (e.g., immunoconjugates or radioimmunoconjugates) comprising antibodies or antigen-binding fragments that are variants of the antibodies shown in Table 1, in that such antibodies or antigen-binding fragments have CDR sequences that differ by no more than two amino acid residues (e.g., two or one amino acid residue(s)) per CDR from the CDR sequences of an antibody described in Table 1. In some embodiments, provided are compounds (e.g., immunoconjugates or radioimmunoconjugates) comprising antibodies or antigen-binding fragments that are variants of the antibodies shown in Table 1, in that such antibodies or antigen-binding fragments have a set of six CDRs whose sequences collectively differ by no more than two amino acid residues (e.g., two or one amino acid residues) from the CDRs of an antibody described in Table 1.

In some embodiments, provided are compounds (e.g., immunoconjugates or radioimmunoconjugates) comprising antibodies or antigen-binding fragments that comprise a heavy chain variable domain and a light chain variable domain which comprise heavy chain variable domain and light chain variable sequences of an antibody described in Table 1. In some embodiments, provided are compounds (e.g., immunoconjugates or radioimmunoconjugates) comprising antibodies or antigen-binding fragments that are variants of the antibodies shown in Table 1, in that such antibodies or antigen-binding fragments have (1) a heavy chain domain comprising an amino acid sequence that is at least 85%, at least 87.5%, at least 91%, at least 91%, at least 92%, at least 93%, at least 94%, at least 95%, at least 96%, at least 97%, at least 98%, or at least 99% identical to the amino acid sequence of the heavy chain variable domain of an antibody described in Table 1; and (2) a light chain domain comprising an amino acid sequence that is at least 85%, at least 87.5%, at least 90%, at least 91%, at least 92%, at least 93% at least 94% at least 95% at least 96%, at least 97% at least 98%, or at least 99% identical to the amino acid sequence of a light chain variable domain of the same antibody described in Table 1.

TABLE 1
Exemplary heavy chain variable domain, light chain variable domain, and
complementarity-determining region sequences of Claudin 18.2 antibodies

| CLDN18 | | |
|---|---|---|
| Antibody | Heavy chain variable domain | Light chain variable domain |
| YU574-D03 | EVQLLESGGGLVQPGGSLRLSCAASG | DIVMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAGRQVAWL | QPEDFATYYCQQANSFPLTFGGGTKV |
| | GNWGQGTLVTVSS | DIK |
| | (SEQ ID NO: 1) | (SEQ ID NO: 5) |
| | CDR-H1: SYAMS | CDR-L1: RASQGISSWLA |
| | (SEQ ID NO: 2) | (SEQ ID NO: 6) |
| | CDR-H2: AISGSGGSTYYADSVKG | CDR-L2: AASSLQS |
| | (SEQ ID NO: 3) | (SEQ ID NO: 7) |
| | CDR-H3: RQVAWLGN | CDR-L3: QQANSFPLT11 |
| | (SEQ ID NO: 4) | (SEQ ID NO: 8) |
| YU574-D11 | QVQLVQSGGGLVQPGGSLRLSCAASG | QSVLTQPPSVSGAPGQRVTISCTGSS |
| | FTFSSYAMSWVRQAPGKGLEWVSAIS | SNIGAGYDVHWYQQLPGTAPKLLIYG |
| | GSGGSTYYADSVKGRFTISRDNSKNT | NSNRPSGVPDRFSGSKSGTSASLAIT |
| | LYLQMNSLRAEDTAVYYCAGGANGFD | GLQAEDEADYYCQSYDSSLSGLVFGG |
| | PWGQGTLVTVSS | GTKLTVL |
| | (SEQ ID NO: 9) | (SEQ ID NO: 13) |
| | CDR-H1: SYAMS | CDR-L1: TGSSSNIGAGYDVH |
| | (SEQ ID NO: 10) | (SEQ ID NO: 14) |
| | CDR-H2: AISGSGGSTYYADSVKG | CDR-L2: GNSNRPS |
| | (SEQ ID NO: 11) | (SEQ ID NO: 15) |
| | CDR-H3: GANGFDP | CDR-L3: QSYDSSLSGLV |
| | (SEQ ID NO: 12) | (SEQ ID NO: 16) |
| YU574-E07 | QVQLQESGGGVVQPGRSLRLSCAASG | QSVLTQPPSASGAPGQRVTISCTGSS |
| | FTFSSYGMHWVRQAPGKGLEWVAVIS | SNIGAGYDVHWYQQLPGTAPKLLIYG |
| | YDGSNKYYADSVKGRFTISRDNSKNT | NSNRPSGVPDRFSGSKSGTSASLAIT |
| | LYLQMNSLRAEDTAVYYCAKDQRGGL | GLQAEDEADYYCQSYDSSLSGWVFGG |
| | YGMDVWGQGTLVTVSS | GTKLTVL |
| | (SEQ ID NO: 17) | (SEQ ID NO: 21) |
| | CDR-H1: SYGMH | CDR-L1: TGSSSNIGAGYDVH |
| | (SEQ ID NO: 18) | (SEQ ID NO: 22) |
| | CDR-H2: VISYDGSNKYYADSVKG | CDR-L2: GNSNRPS |
| | (SEQ ID NO: 19) | (SEQ ID NO: 23) |
| | CDR-H3: DQRGGLYGMDV | CDR-L3: QSYDSSLSGWV |
| | (SEQ ID NO: 20) | (SEQ ID NO: 24) |
| YU574-H09 | QVQLVQSGAEVKKPGASVKVSCKASG | SYVLTQPPSASGTPGQRVTISCSGSS |
| | YTFTSYYMHWVRQAPGQGLEWMGIIN | SNIGSNYVYWYQQLPGTAPKLLIYSN |
| | PSGGSTSYAQKFQGRVTMTRDTSTST | NQRPSGVPDRFSGSKSGTSASLAISG |
| | VYMELSSLRSEDTAVYYCARGGSGFD | LRSEDEADYYCAAWDDSLSGWVFGGG |
| | YWGQGTLVTVSS | TKLTVL |
| | (SEQ ID NO: 25) | (SEQ ID NO: 29) |
| | CDR-H1: SYYMH | CDR-L1: SGSSSNIGSNYVY |
| | (SEQ ID NO: 26) | (SEQ ID NO: 30) |
| | CDR-H2: IINPSGGSTSYAQKFQG | CDR-L2: SNNQRPS |
| | (SEQ ID NO: 27) | (SEQ ID NO: 31) |
| | CDR-H3: GGSGFDY | CDR-L3: AAWDDSLSGWV |
| | (SEQ ID NO: 28) | (SEQ ID NO: 32) |
| YU575-H03 | EVQLVQSGGGLVQPGGSLRLSCAASG | QSVLTQPPSVSGAPGQRVTISCTGSS |
| | FTFSSYAMSWVRQAPGKGLEWVSAIS | SNIGAGYDVHWYQQLPGTAPKLLIYG |
| | GSGGSTYYADSVKGRFTISRDNSKNT | NSNRPSGVPDRFSGSKSGTSASLAIT |
| | LYLQMNSLRAEDTAVYYCAIPGDYGS | GLQAEDEADYYCQSYDSSLSGYVFGT |
| | SDPWGQGTLVTVSS | GTKLTVL |
| | (SEQ ID NO: 33) | (SEQ ID NO: 37) |
| | CDR-H1: SYAMS | CDR-L1: TGSSSNIGAGYDVH |
| | (SEQ ID NO: 34) | (SEQ ID NO: 38) |
| | CDR-H2: AISGSGGSTYYADSVKG | CDR-L2: GNSNRPS |
| | (SEQ ID NO: 35) | (SEQ ID NO: 39) |
| | CDR-H3: PGDYGSSDP | CDR-L3: QSYDSSLSGYV |
| | (SEQ ID NO: 36) | (SEQ ID NO: 40) |
| YU577-B01 | EVQLVESGGGVVQPGRSLRLSCAASG | DIQLTQSPSSLSASVGDRVTITCRAS |
| | FTFSSYGMHWVRQAPGKGLEWVAVIS | QGISNYLAWFQQKPGKAPKSLIYAAS |
| | YDGSNKYYADSVKGRFTISRDNSKNT | SLQSGVPSKFSGSGSGTDFTLTISSL |
| | LYLQMNSLRAEDTAVYYCAKDVTYYY | QPEDFATYYCQQYNSYPYSFGQGTKV |
| | DSSGSPDFDYWGQGTLVTVSS | EIK |
| | (SEQ ID NO: 41) | (SEQ ID NO: 45) |
| | CDR-H1: SYGMH | CDR-L1: RASQGISNYLA |
| | (SEQ ID NO: 42) | (SEQ ID NO: 46) |
| | CDR-H2: VISYDGSNKYYADSVKG | CDR-L2: AASSLQS |
| | (SEQ ID NO: 43) | (SEQ ID NO: 47) |
| | CDR-H3: DVTYYYDSSGSPDFDY | CDR-L3: QQYNSYPYS |
| | (SEQ ID NO: 44) | (SEQ ID NO: 48) |
| YU577-B11 | EVQLVESGGGLVQPGGSLRLSCAASG | DIQMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAGYTGGAF | QPEDFATYYCQQANSFPLTFGGGTKL |
| | DIWGQGTMVTVSS | EIK |
| | (SEQ ID NO: 49) | (SEQ ID NO: 53) |
| | CDR-H1: SYAMS | CDR-L1: RASQGISSWLA |
| | (SEQ ID NO: 50) | (SEQ ID NO: 54) |
| | CDR-H2: AISGSGGSTYYADSVKG | CDR-L2: AASSLQS |
| | (SEQ ID NO: 51) | (SEQ ID NO: 55) |
| | CDR-H3: YTGGAFDI | CDR-L3: QQANSFPLT |
| | (SEQ ID NO: 52) | (SEQ ID NO: 56) |
| YU577-D10 | QVQLVQSGGGLVQPGGSLRLSCAASG | DIQMTQSPSSLSASVGDRVTITCQAS |
| | FTFSSYAMSWVRQAPGKGLEWVSAIS | QDISNYLNWYQQKPGKAPKLLIYDAS |
| | GSGGSTYYADSVKGRFTISRDNSKNT | NLETGVPSRFSGSGSGTDFTFTISSL |
| | LYLQMNSLRAEDTAVYYCAKPLRFLG | QPEDIATYYCQQYDNLPLTFGGGTKL |
| | DYGGWGQGTLVTVSS | EIK |
| | (SEQ ID NO: 57) | (SEQ ID NO: 61) |
| | CDR-H1: SYAMS | CDR-L1: QASQDISNYLN |
| | (SEQ ID NO: 58) | (SEQ ID NO: 62) |
| | CDR-H2: AISGSGGSTYYADSVKG | CDR-L2: DASNLET |
| | (SEQ ID NO: 59) | (SEQ ID NO: 63) |
| | CDR-H3: PLRFLGDYGG | CDR-L3: QQYDNLPLT |
| | (SEQ ID NO: 60) | (SEQ ID NO: 64) |
| YU577-E08 | QVQLVQSGAEVKKPGASVKVSCKASG | QSVLTQPPSVSAAPGQKVTISCSGSS |
| | YTFTSYYMHWVRQAPGQGLEWMGIIN | SNIGNNYVSWYQQLPGTAPKLLIYEN |
| | PSGGSTSYAQKFQGRVTMTRDTSTST | NKRPSGIPDRFSGSKSGTSATLGITG |
| | VYMELSSLRSEDTAVYYCATRQFDYW | LQTGDEADYYCGTWDSSLSAAVFGGG |
| | GQGTLVTVSS | TKVTVL |
| | (SEQ ID NO: 65) | (SEQ ID NO: 69) |
| | CDR-H1: SYYMH | CDR-L1: SGSSSNIGNNYVS |
| | (SEQ ID NO: 66) | (SEQ ID NO: 70) |
| | CDR-H2: IINPSGGSTSYAQKFQG | CDR-L2: ENNKRPS |
| | (SEQ ID NO: 67) | (SEQ ID NO: 71) |
| | CDR-H3: RQFDY | CDR-L3: GTWDSSLSAAV |
| | (SEQ ID NO: 68) | (SEQ ID NO: 72) |
| YU577-E12 | QVQLVESGGGLVQPGGSLRLSCAASG | DIVMTQSPSFLSASAGDRVTITCRAS |
| | FTFSSYAMSWVRQAPGKGLEWVSAIS | QGISSYLAWYQQKPGKGPKLLIYAAS |
| | GSGGSTYYADSVKGRFTISRDNSKNT | TLQSGVPSRFSGSGSGTEFTLTISSL |
| | LYLQMNSLRAEDTAVYYCAKDLGVYY | QPEDFATYYCQQVNSYPLTFGPGTKV |
| | FDYWGQGTLVTVSS | DIK |
| | (SEQ ID NO: 73) | (SEQ ID NO: 77) |
| | CDR-H1: SYAMS | CDR-L1: RASQGISSYLA |
| | (SEQ ID NO: 74) | (SEQ ID NO: 78) |
| | CDR-H2: AISGSGGSTYYADSVKG | CDR-L2: AASTLQS |
| | (SEQ ID NO: 75) | (SEQ ID NO: 79) |
| | CDR-H3: DLGVYYFDY | CDR-L3: QQVNSYPLT |
| | (SEQ ID NO: 76) | (SEQ ID NO: 80) |
| YU577-H07 | EVQLLESGGGLVQPGGSLRLSCAASG | DIQMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAKEPFYGM | QPEDFATYYCQQANSFPLTFGGGTKL |
| | DVWGQGTLVTVSS | EIK |
| | (SEQ ID NO: 81) | (SEQ ID NO: 85) |
| | CDR-H1: SYAMS | CDR-L1: RASQGISSWLA |
| | (SEQ ID NO: 82) | (SEQ ID NO: 86) |
| | CDR-H2: AISGSGGSTYYADSVKG | CDR-L2: AASSLQS |
| | (SEQ ID NO: 83) | (SEQ ID NO: 87) |
| | CDR-H3: EPFYGMDV | CDR-L3: QQANSFPLT |
| | (SEQ ID NO: 84) | (SEQ ID NO: 88) |
| YU578-A02 | EVQLLESGGGLVQPGGSLRLSCAASG | DIVMTQSPSTLSASVGDRVTITCRAS |
| | FTFSSYAMSWVRQAPGKGLEWVSAIS | QSISSWLAWYQQKPGKAPKLLIYKAS |
| | GSGGSTYYADSVKGRFTISRDNSKNT | SLESGVPSRFSGSGSGTEFTLTISSL |
| | LYLQMNSLRAEDTAVYYCAKPPGSYY | QPDDFATYYCQQYNSYPLTFGGGTKV |
| | VWDYWGQGTLVTVSS | EIK |
| | (SEQ ID NO: 89) | (SEQ ID NO: 93) |
| | CDR-H1: SYAMS | CDR-L1: RASQSISSWLA |
| | (SEQ ID NO: 90) | (SEQ ID NO: 94) |
| | CDR-H2: AISGSGGSTYYADSVKG | CDR-L2: KASSLES |
| | (SEQ ID NO: 91) | (SEQ ID NO: 95) |
| | CDR-H3: PPGSYYVWDY | CDR-L3: QQYNSYPLT |
| | (SEQ ID NO: 92) | (SEQ ID NO: 96) |
| YU578-D01 | EVQLVQSGGGLVQPGGSLRLSCAASG | DIQLTQSPSSLSASVGDRVTITCRAS |
| | FTFSSYAMSWVRQAPGKGLEWVSAIS | QGISNYLAWFQQKPGKAPKSLIYAAS |
| | GSGGSTYYADSVKGRFTISRDNSKNT | SLQSGVPSKFSGSGSGTDFTLTISSL |
| | LYLQMNSLRAEDTAVYYCAKTAGSYY | QPEDFATYYCQQYNSYPLTFGGGTKV |
| | ILDYWGQGTLVTVSS | DIK |
| | (SEQ ID NO: 97) | (SEQ ID NO: 101) |
| | CDR-H1: SYAMS | CDR-L1: RASQGISNYLA |
| | (SEQ ID NO: 98) | (SEQ ID NO: 102) |
| | CDR-H2: AISGSGGSTYYADSVKG | CDR-L2: AASSLQS |
| | (SEQ ID NO: 98) | (SEQ ID NO: 103) |
| | CDR-H3: TAGSYYILDY | CDR-L3: QQYNSYPLT |
| | (SEQ ID NO: 100) | (SEQ ID NO: 104) |
| YU578-F02 | QVQLVESGGGLVQPGGSLRLSCAASG | DIVMTQSPSFLSASVGDRVTITCRAS |
| | FTFSSYAMSWVRQAPGKGLEWVSAIS | QGISSYLAWYQQKPGKAPKLLIYAAS |
| | GSGGSTYYADSVKGRFTISRDNSKNT | TLQSGVPSRFSGSGSGTEFTLTISSL |
| | LYLQMNSLRAEDTAVYYCAKPLTAGY | QPEDFATYYCQQLNSYPITFGQGTKL |
| | YYGMVAFDIWGQGTMVTVSS | EIK |
| | (SEQ ID NO: 105) | (SEQ ID NO: 109) |
| | CDR-H1: SYAMS | CDR-L1: RASQGISSYLA |
| | (SEQ ID NO: 106) | (SEQ ID NO: 110) |
| | CDR-H2: AISGSGGSTYYADSVKG | CDR-L2: AASTLQS |
| | (SEQ ID NO: 107) | (SEQ ID NO: 111) |
| | CDR-H3: PLTAGYYYGMVAFDI | CDR-L3: QQLNSYPIT |
| | (SEQ ID NO: 108) | (SEQ ID NO: 112) |

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A reference polypeptide described herein can include a target-binding domain that is capable of binding to a target of interest (e.g., is capable of binding to an antigen, e.g., human CLDN 18.2). For example, a polypeptide, such as an antibody, can bind to a transmembrane polypeptide (e.g., receptor) or ligand (e.g., a growth factor).

### Modified Polypeptides

Polypeptides suitable for use with compositions and methods of the present disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to human CLDN 18.2). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide herein can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 2. If such substitutions result in a change not desired, then other type of substitutions, denominated “exemplary substitutions” in Table 2, or as further described herein in reference to amino acid classes, are introduced and the products screened.

TABLE 2
Amino acid substitutions

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain.

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α,α′, α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaoxacyclohexadecane-1,4,7,10,13,16-hexaacetic acid), PEPA (1,4,7,10,13-pentaazacyclopentadecane-N,N′,N″,N′″, N″″-pentaacetic acid), H₄ octapa (N,N′-bis(6-carboxy-2-pyridylmethyl)-ethylenediamine-N,N′-diacetic acid), H₂ dedpa (1,2-[[6-(carboxy)-pyridin-2-yl]-methylamino]ethane), H₆ phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″, N′″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), or porphyrins.

Preferably, the chelating moiety is selected from DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, compounds comprise a metal complex of a chelating moiety. For example, chelating groups may be used in metal chelate combinations with metals, such as manganese, iron, and gadolinium and isotopes (e.g., isotopes in the general energy range of 60 to 10,000 keV), such as any of the radioisotopes and radionuclides discussed herein.

In some embodiments, chelating moieties are useful as detection agents, and compounds comprising such detectable chelating moieties can therefore be used as diagnostic or theranostic agents.

In some embodiments, variable A of Formula I is a macrocyclic chelating moiety comprising one or more heteroaryl groups (e.g., six-membered nitrogen-containing heteroaryl). Examples of such macrocyclic chelating moiety include, but are not limited to:

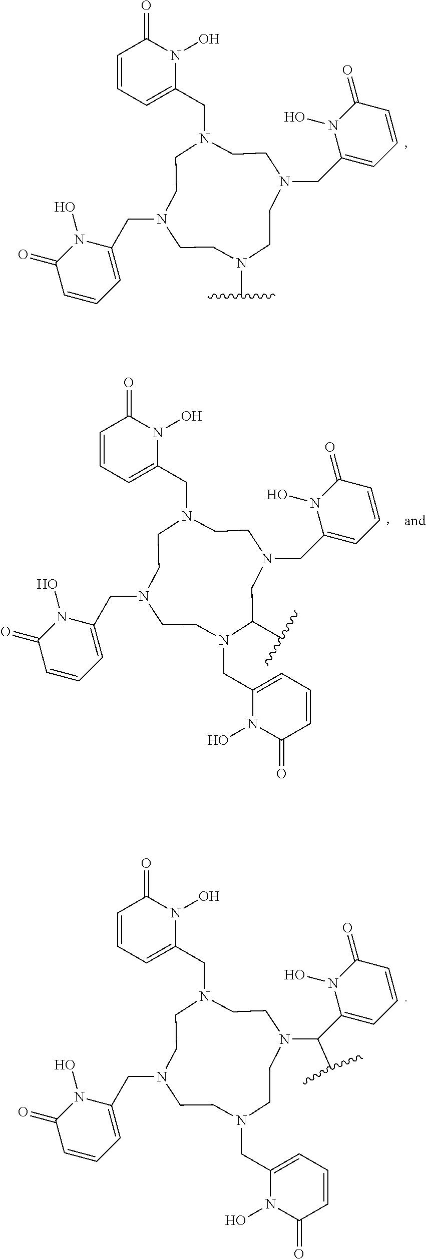 [SMILES: *N1CCN(Cc2cccc(=O)n2O)CCN(Cc2cccc(=O)n2O)CCN(Cc2cccc(=O)n2O)CC1 ; *C1CN(Cc2cccc(=O)n2O)CCN(Cc2cccc(=O)n2O)CCN(Cc2cccc(=O)n2O)CCN1Cc1cccc(=O)n1O ; *C(c1cccc(=O)n1O)N1CCN(Cc2cccc(=O)n2O)CCN(Cc2cccc(=O)n2O)CCN(Cc2cccc(=O)n2O)CC1]

### Radioisotopes and Radionuclides

In some embodiments, the metal complex comprises a radionuclide. Examples of suitable radioisotopes and radionuclides include, but are not limited to, ³H, ¹⁴C, ¹⁵N, ¹⁸F, ³⁵S ⁴⁴Sc, ⁴⁷Sc, ⁵⁵Co, ⁶⁰Cu, ⁶¹Cu, ⁶²Cu, ⁶⁴Cu, ⁶⁶Ga, ⁶⁷Ga, ⁶⁷Cu, ⁶⁸Ga, ⁷⁵Br, ⁷⁶Br, ⁷⁷Br, ⁸²Rb, ⁸⁹Zr, ⁸⁶Y, ⁸⁷Y, ⁹⁰Y⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, the metal complex comprises a radionuclide selected from ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th. In certain embodiments, the metal complex comprises a radionuclide selected from ⁶⁸Ga, ⁸⁹Zr, ⁹⁰Y, ¹¹¹In, ¹⁷⁷Lu, and ²²⁵Ac. In certain embodiments, the metal complex comprises a radionuclide being ¹⁷⁷Lu or ²²⁵Ac.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), or Terbium-149 (¹⁴⁹Tb), or a progeny thereof. In some embodiments, the alpha-emitter is Actinium-225 (²²⁵Ac), or a progeny thereof.

### Linker

The compounds of this invention comprise the structure of Formula I below:

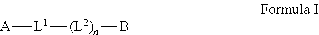

wherein each of the variables is defined in the SUMMARY section above.

Each of the compounds of Formula I comprises a linker moiety as -L¹-(L²)ₙ-, wherein:
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
each L², independently, has the structure:

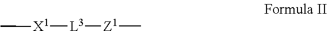

wherein:
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl (e.g., (CH₂CH₂O)₂₋₂₀); and
Z¹ is —CH₂— #, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

In some embodiments, L¹ is optionally substituted C₁-C₆ alkyl or optionally substituted C₁-C₆ heteroalkyl. In certain embodiments, L¹ is substituted C₁-C₆ alkyl or substituted C₁-C₆ heteroalkyl, the substituent comprising a heteroaryl group (e.g., six-membered nitrogen-containing heteroaryl). In some embodiments, L¹ is C₁-C₆ alky. For example, L¹ is —CH₂CH₂—. In some embodiments, L¹ is a bond. In some embodiments, L¹ is

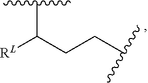

wherein R^{L} is hydrogen or —CO₂H.

In some embodiments, X¹ is —C(O)NR¹—*, —NR¹C(O)—*, or —NR¹—, “*” indicating the attachment point to L³, and R¹ is hydrogen, optionally substituted C₁-C₆ alkyl, optionally substituted C₁-C₆ heteroalkyl, optionally substituted aryl, or optionally substituted heteroaryl. In some embodiments, X¹ is —C(O)NR¹—*, “*” indicating the attachment point to L³, and R¹ is hydrogen.

In some embodiments, L³ is optionally substituted C₁-C₅₀ alkyl (e.g., C₃-C₃₀ alkyl, C₃-C₂₅ alkyl, C₃-C₂₀ alkyl, C₃-C₁₅ alkyl, C₃-C₁₀ alkyl, C₅-C₃₀ alkyl, C₅-C₂₅ alkyl, C₅-C₂₀ alkyl, C₅-C₁₅ alkyl, and C₅-C₁₀ alkyl) or optionally substituted C₁-C₅₀ heteroalkyl (e.g., C₃-C₃₀ heteroalkyl, C₃-C₂₅ heteroalkyl, C₃-C₂₀ heteroalkyl, C₃-C₁₅ heteroalkyl, C₃-C₁₀ heteroalkyl, C₅-C₃₀ heteroalkyl, C₅-C₂₅ heteroalkyl, C₅-C₂₀ heteroalkyl, C₅-C₁₅ heteroalkyl, and C₅-C₁₀ heteroalkyl). An exemplary C₁-C₅₀ heteroalkyl is C₅-C₃₀ polyethylene glycol (e.g., C₅-C₂₅ polyethylene glycol, C₅-C₂₀ polyethylene glycol, C₅-C₁₅ polyethylene glycol).

In certain embodiments, L³ is C₅-C₂₅ polyethylene glycol, C₅-C₂₀ polyethylene glycol, or C₅-C₁₅ polyethylene glycol.

In some embodiments, L³ is optionally substituted C₁-C₅₀ heteroalkyl (e.g., C₁-C₄₀ heteroalkyl, C₁-C₃₀ heteroalkyl, C₁-C₂₀ heteroalkyl, C₂-C₁₈ heteroalkyl, C₃-C₁₆ heteroalkyl, C₄-C₁₄ heteroalkyl, C₅-C₁₂ heteroalkyl, C₆-C₁₀ heteroalkyl, C₈-C₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl).

In some embodiments, L³ is optionally substituted C₁-C₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L³ is optionally substituted C₁-C₅₀ heteroalkyl comprising a polyethylene glycol (PEG) moiety comprising 1-20 oxyethylene (—O—CH₂—CH₂—) units or portions thereof. For example, L³ comprises PEG3 as shown below:

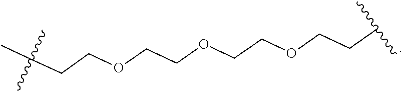 [SMILES: *CCOCCOCCOCC*]

In some embodiments, L³ is (CH₂CH₂O)ₘ(CH₂)_{w}, and m and w are each independently an integer between 0 and 10 (inclusive), and at least one of m and w is not 0.

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, Z¹ is CH₂, C═O, or NR¹; wherein R¹ is H, optionally substituted C₁-C₆ alkyl, optionally substituted C₁-C₆ heteroalkyl, optionally substituted aryl, or optionally substituted heteroaryl.

In certain embodiments, A-L¹-(L²)ₙ-B can be represented by the following structure:

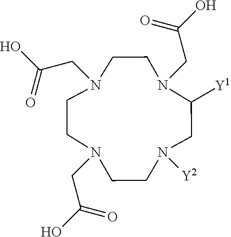

wherein Y¹ is —CH₂OCH₂(L²)ₙ-B, C═O(L²)ₙ-B, or C═S(L²)ₙ-B and Y² is —CH₂CO₂H; or wherein Y¹ is H and Y² is L¹-(L²)ₙ-B.

### Cross-Linking Groups

In some embodiments, compounds (e.g., radioimmunoconjugates) are synthesized using bifunctional chelates that comprise a chelate, a linker, and a cross-linking group. Once the compound (e.g., radioimmunoconjugate) is formed, the cross-linking group may be absent from the compound (e.g., radioimmunoconjugate).

In some embodiments, compounds (e.g., radioimmunoconjugates) comprise a cross-linking group instead of or in addition to the targeting moiety (e.g., in some embodiments, B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a comprises sortase recognition sequence. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Pharmaceutical Compositions

In one aspect, the present disclosure provides pharmaceutical compositions comprising compounds disclosed herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, PA, 17th ed., 1985. For a brief review of methods for drug delivery, See, e.g., Langer (Science. 249:1527-1533, 1990).

Pharmaceutical compositions may be formulated for any of a variety of routes of administration discussed herein (See, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Methods of Treatment

In one aspect, the present disclosure provides methods of treatment comprising administering to a subject in need thereof a compound (e.g., radioimmunoconjugate) as disclosed herein.

### Subjects

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases. Provided compounds (e.g., radioimmunoconjugates) and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is any cancer that comprises cells expressing CLDN 18.2. In certain embodiments, the cancer is glioblastoma multiforme or carcinoma.

### Administration and Dosage

Compounds (e.g., radioimmunoconjugates) and pharmaceutical compositions thereof disclosed herein may be administered by any of a variety of routes of administration, including systemic and local routes of administration

Systemic routes of administration include parenteral routes and enteral routes. In some embodiments, compounds (e.g., radioimmunoconjugates) or pharmaceutical compositions thereof are administered by a parenteral route, for example, intravenously, intraarterially, intraperitoneally, subcutaneously, intracranially, or intradermally. In some embodiments, compounds (e.g., radioimmunoconjugates) or pharmaceutical compositions thereof are administered intravenously. In some embodiments, compounds (e.g., radioimmunoconjugates) or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the compound (e.g., radioimmunoconjugate) may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a compound (e.g., radioimmunoconjugate) or composition in an amount effective for radiation treatment planning, then administered a second dose or set of doses of the compound (e.g., radioimmunoconjugate) or composition in a therapeutically effective amount.

For treating cancer comprising cells expressing CLDN 18.2, the method of this invention typically comprises administering to a subject (e.g., a human) in need thereof a first dose of a compound or composition provided above in an amount effective for radiation treatment planning, followed by administering subsequent doses of a compound or composition provided above in a therapeutically effective amount.

In some embodiments, the compound or composition administered in the first dose and the compound or composition administered in the second dose are the same.

In some embodiments, the compound or composition administered in the first dose and the compound or composition administered in the second dose are different.

Therapeutically effective amounts may depend on the severity of the disease or condition and other characteristics of the subject (e.g., weight). Therapeutically effective amounts of disclosed compounds (e.g., radioimmunoconjugates) and compositions for subjects (e.g., mammals such as humans) can be determined by the ordinarily-skilled artisan with consideration of individual differences (e.g., differences in age, weight and the condition of the subject).

In some embodiments, disclosed compounds (e.g., radioimmunoconjugates) exhibit an enhanced ability to target cancer cells. In some embodiments, the effective amount of disclosed compounds (e.g., radioimmunoconjugates) is lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose for a therapeutic effect of the unconjugated, and/or non-radiolabeled targeting moiety.

Single or multiple administrations of pharmaceutical compositions disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

The following specific examples are to be construed as merely illustrative, and not limitative of the remainder of the disclosure in any way whatsoever.

### EXAMPLES

### Example 1. General Materials and Methods

Lutetium-177 can be obtained from ITM Medical Isotopes as lutetium trichloride in a 0.05N hydrochloric acid solution; indium-111, as indium trichloride in a 0.05N hydrochloric acid solution, can be obtained from BWXT; and actinium-225 can be obtained as actinium-225 trinitrate from Oak Ridge National Laboratories or actinium-225 trichloride from Canadian Nuclear Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 m) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 m) column.

HPLC elution method 1: Waters Acquity BEH C18 2.1×50 mm (1.7 m) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 m) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 m) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 m) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 m) column; mobile phase A: H₂O (0.1% v/v TFA); mobile phase B: acetonitrile (0.1% v/v TFA); flow rate: 10 mL/min; initial=90% A, 3 min=90% A, 13 min=0% A, 20 min=0% A.

HPLC elution method 6: Waters XBridge Prep C18 OBD 19×100 mm (5 m) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 m) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 m) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radio detector (FC-3300) and TOSOH TSKgel G3000SWxl, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radioTLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

Generation and evaluation of certain CLDN 18.2 monoclonal antibodies can follow the protocols provided below.

A naïve human antibody library can be used to select CLDN18.2 antibodies, using CLDN18.2 antigen or CLDN18.2-expressing cells for biopanning. ELISA and/or flow cytometry methods can be used to screen for antibody clones with human and mouse CLDN18.2 affinity but no human CLDN18.1 cross-reactivity. In-silico developability can be assessed by constructing 3D models of the antibodies and calculating key characteristics, including isoelectric point (pI), germinality index (GI), unusual residues, CDR length, hydrophobicity score, charge, amidation site, and glycosylation pattern. The characteristics obtained from the in-silico developability assessment can be compared with therapeutic antibodies and human antibody databases to rank the developability of the putative antibody clones. Variable domain sequences of selected clone can then be constructed into a human IgG4 S228P expression vector and transiently transfected into HEK cells for antibody production. Expressed antibodies can be purified via Protein A affinity chromatography, followed by UV-Vis spectroscopic and SDS-PAGE analyses.

Purified monoclonal antibodies can be further assessed for CLDN18.2 binding ability and non-specific/off-target binding using ELISA and/or flow cytometric methods. Additional characterization, such as antibody internalization and stability, can be performed using methods known in the art.

### Example 2. Synthesis of 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic Acid (Compound B)

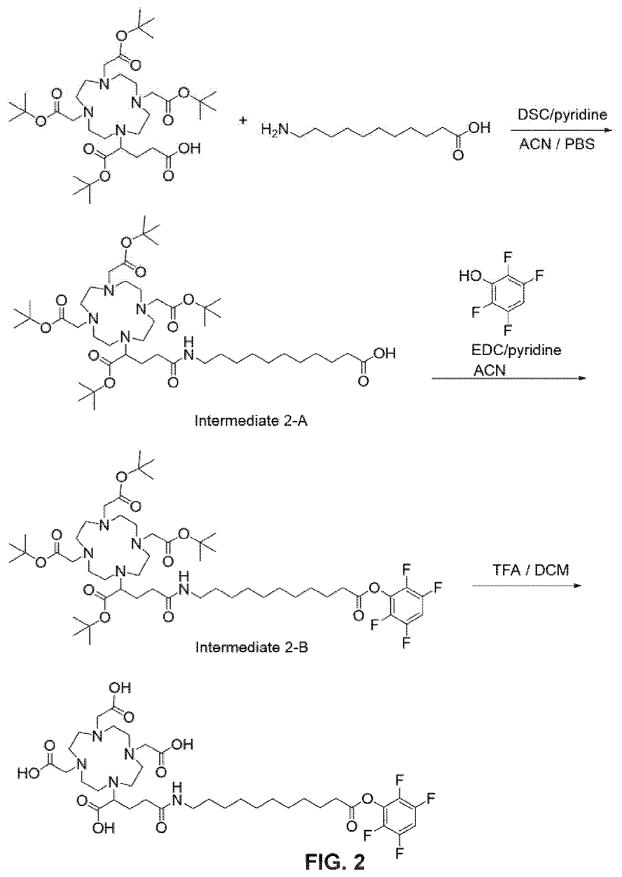

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 2. To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA-(tBu)₄, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHz, DMSO-d₆) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 3. Synthesis of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic Acid (Compound C)

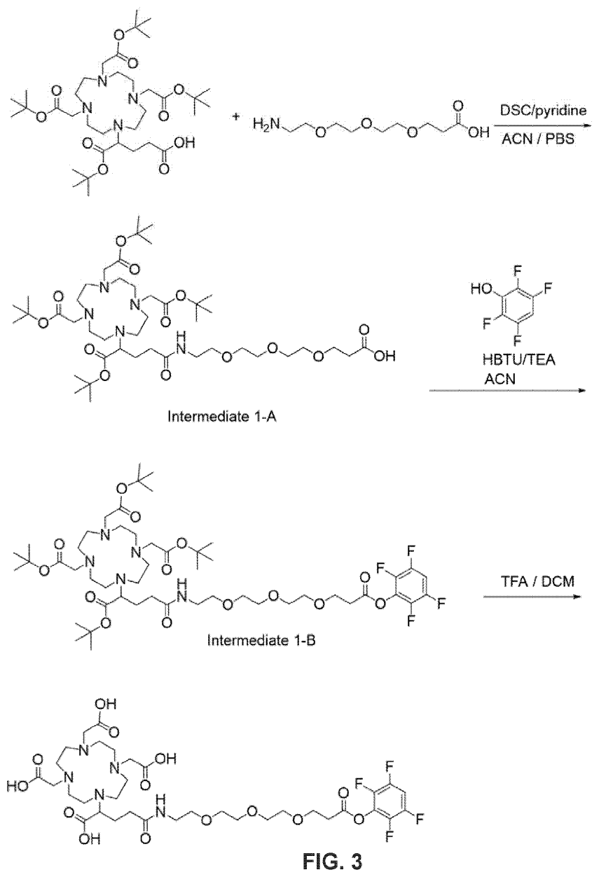

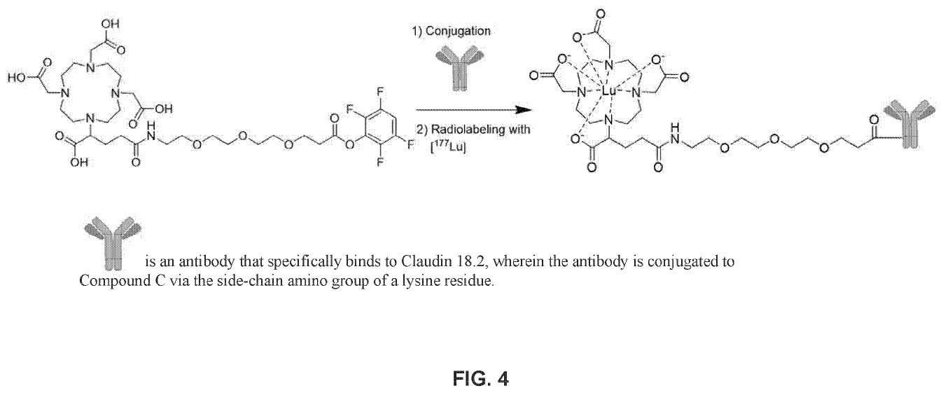

A bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 3.

To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA(tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 mol) is added ACN (2 mL), NEt₃ (50 μL, 360 μmol, 6 equiv.), HBTU (23 mg, 60 mol, 1 equiv) and a TFP solution (50 mg, 300 mol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 mol) is added DCM (2 mL) and TFA (2 mL). The resulting solution is stirred at ambient temperature for 16 hour. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution is then purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d₆, 600 MHz) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 4. Conjugation and Radiolabeling for Synthesis of Radioimmunoconjugates Comprising Claudin 18.2 Antibodies

### 4.1. Synthesis of Immunoconjugate Compound D

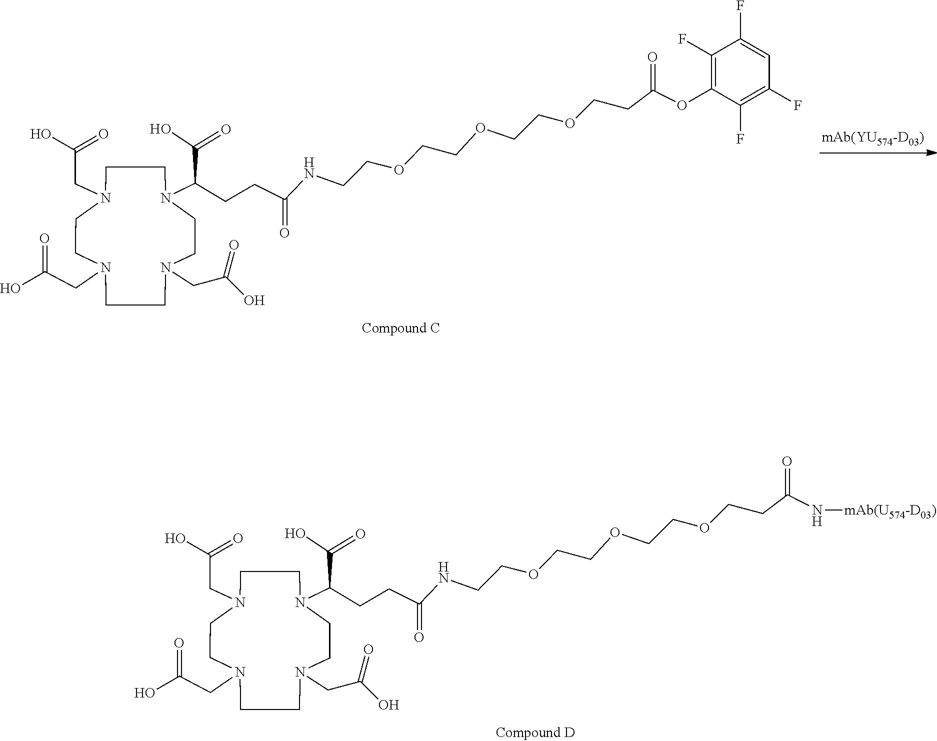

A 1.5 mL Eppendorf tube was charged with a SABST (sodium acetate buffered saline with tween) solution of YU574-D03 (0.54 mL; 5.555 mg/mL; 3.00 mg), followed by 54 μL carbonate buffer (pH 9.5) and 30.4 μL of a 0.001 M HCl solution of Compound C (R enantiomer; 6 equiv.; 1.25 mg in 300 μL; this solution was prepared immediately prior to use). The reaction was allowed to react for ca. one hour at room temperature; the pH of the resulting mixture was ca. 9.5-10 by pH strip. The reaction mixture (ca. 600 μL) was purified using 1 mL columns packed with G50 resin (hydrated with SABST). The product fractions were eluted using SABST and combined to afford a clear colourless liquid, wherein analysis using Size Exclusion Chromatography-High Performance Liquid Chromatography (SEC-HPLC) indicated the formation of Compound D (0.9 mL, 3.202 mg/mL, 2.86 mg, 95%). A chelate-to-antibody ratio (CAR) of 5.4 was determined using Matrix-Assisted Laser Desorption Ionization Time-of-Flight (MALDI-TOF) Mass Spectrometry, and was calculated from the difference in the mass-to charge ratio between antibody YU574-D03 and immunoconjugate Compound D.

### 4.2 Synthesis of Immunoconjugate Compound E

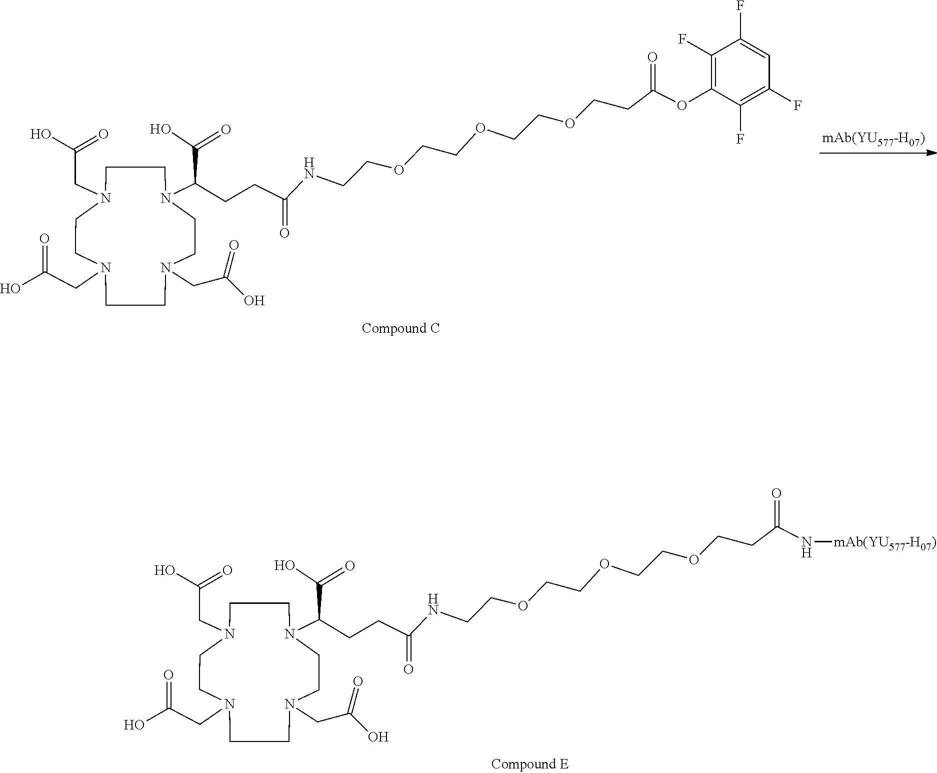

A 1.5 mL Eppendorf tube was charged with a SABST solution of YU577-H07 (0.72 mL; 4.427 mg/mL; 3.19 mg), followed by 72 μL carbonate buffer (pH 9.5) and 27.7 μL of a 0.001 M HCl solution of Compound C (R enantiomer; 7 equiv.; 1.7 mg in 300 μL; this solution was prepared immediately prior to use). The reaction was allowed to react for ca. one hour at room temperature; the pH of the resulting mixture was ca. 9.5-10 by pH strip. The reaction mixture (ca. 800 μL) was purified using 1 mL columns packed with G50 resin (hydrated with SABST). The product fractions were eluted using SABST and combined to afford a clear colourless liquid, wherein analysis using SEC-HPLC indicated the formation of Compound E (1.2 mL, 2.527 mg/mL, 3.00 mg, 94%). A chelate-to-antibody ratio (CAR) of 3.5 was determined using MALDI-TOF Mass Spectrometry, and was calculated from the difference in the mass-to charge ratio between antibody YU577-H07 and immunoconjugate Compound E.

### 4.3 Radiosynthesis of Radioimmunoconjugate Compound F

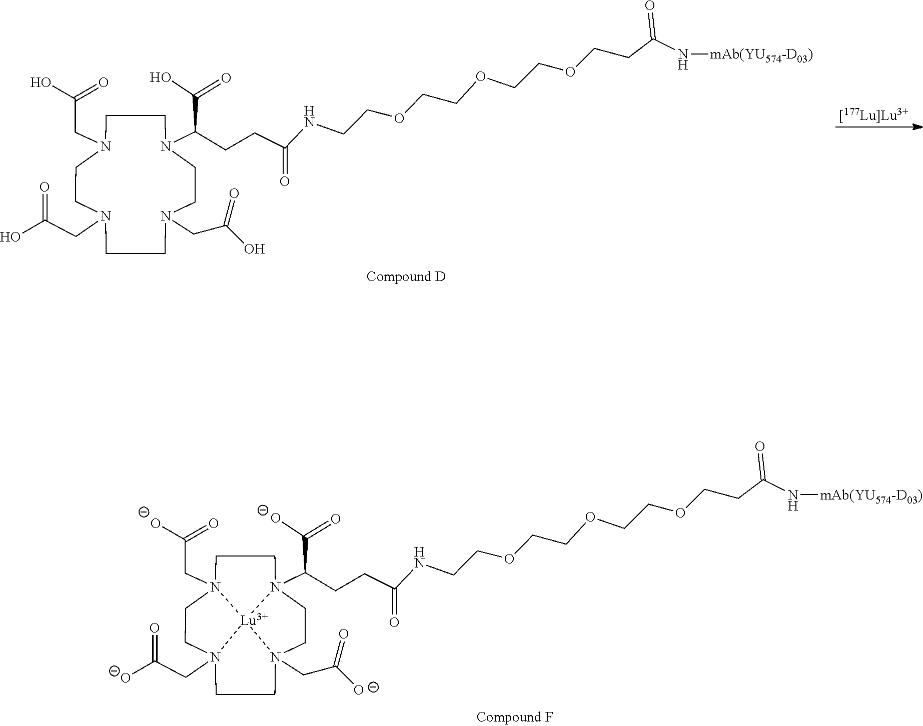

A 1.5 mL Eppendorf tube was charged with a SABST solution of Compound D (0.100 mL; 2.00 mg/mL; 0.200 mg), followed by a 0.04 M HCl solution of [¹⁷⁷Lu]LuCl₃ (ca. 2.25 mCi). After ca. 60 min at room temperature, Instant Thin Layer Chromatography (ITLC) analysis of the reaction mixture (silica gel (SG)-plate, 0.02 M citrate buffer (5% MeOH) as the mobile phase) indicated a radiochemical conversion (RCC) of 96.0%. Purification was carried out using a 1 mL column packed with G50 resin (hydrated with SABST). The product fractions were eluted using SABST and combined. SABST solutions of sodium L-ascorbate and diethylenetriamine-pentaacetic acid calcium trisodium salt hydrate (DTPA) were added, and analysis of the resulting formulation (ca. 10 mM sodium L-ascorbate, 1 mM DTPA) by ITLC and SEC-HPLC at end-of-synthesis (EOS) indicated the formation of Compound F (339 μL, 0.449 mg/mL, 10.7 mCi/mg specific activity, 99.1% radiochemical purity, and 98.7% chemical purity).

### 4.4 Radiosynthesis of Radioimmunoconjugate Compound G

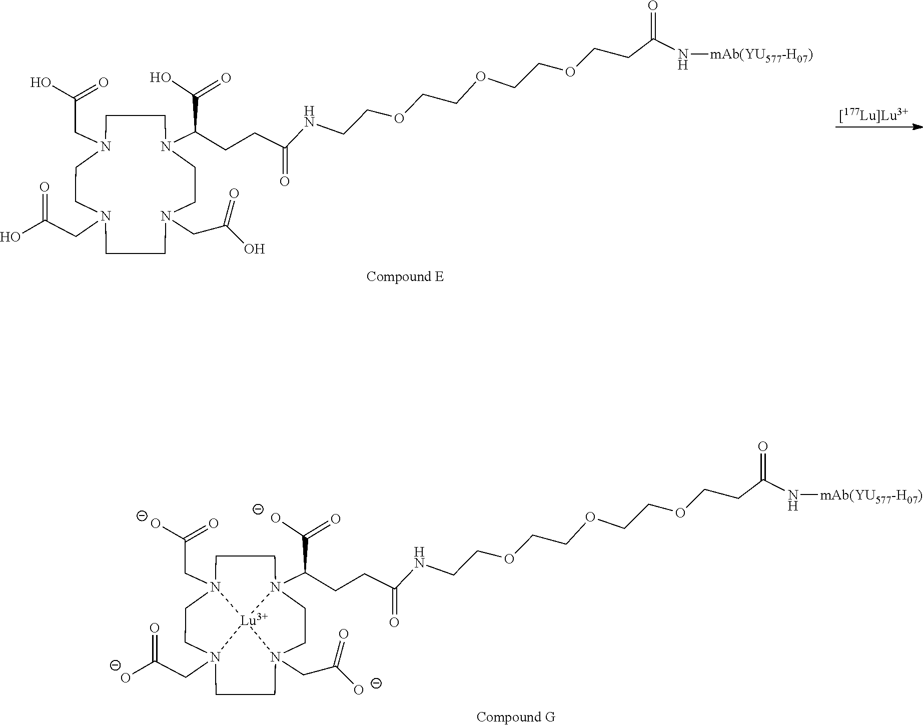

A 1.5 mL Eppendorf tube was charged with a SABST solution of Compound E (0.100 mL; 2.00 mg/mL; 0.200 mg), followed by a 0.04 M HCl solution of [¹⁷⁷Lu]LuCl₃ (ca. 2.37 mCi). After ca. 60 min at room temperature, ITLC analysis of the reaction mixture (SG-plate, 0.02 M citrate buffer (5% MeOH) as the mobile phase) indicated a RCC of 95.7%. Purification was carried out using a 1 mL column packed with G50 resin (hydrated with SABST). The product fractions were eluted using SABST and combined. SABST solutions of sodium L-ascorbate and DTPA were added, and analysis of the resulting formulation (ca. 10 mM sodium L-ascorbate, 1 mM DTPA) by ITLC and SEC-HPLC at EOS indicated the formation of Compound G (364 μL, 0.492 mg/mL, 10.3 mCi/mg specific activity, 99.2% radiochemical purity, and 98.8% chemical purity).

### 4.5 Radiosynthesis of Radioimmunoconjugate Compound H

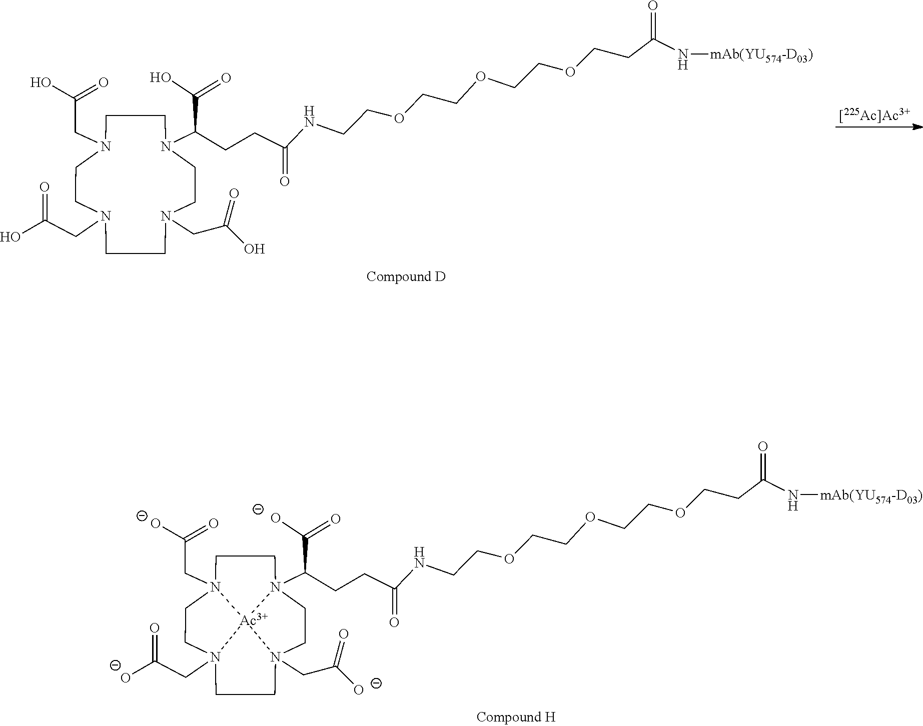

A 1.5 mL Eppendorf tube was charged with a SABST solution of Compound D (0.184 mL; 2.18 mg/mL; 0.400 mg), followed by a 0.001 M HCl solution of [²²⁵Ac]Ac(NO₃)₃ (ca. 22 μCi). After ca. 120 min at 35° C., the reaction was quenched by the addition of DTPA (2 μL of a 0.1 M SABST solution). ITLC analysis of the reaction mixture (SG-plate, 0.02 M citrate buffer (500 MeOH) as the mobile phase) indicated a RCC of 98.0%. Purification was carried out using a 1 mL column packed with G50 resin (hydrated with SABST). The product fractions were eluted using SABST and combined. SABST solutions of sodium L-ascorbate and DTPA were added, and analysis of the resulting formulation (ca. 10 mM sodium L-ascorbate, 1 mM DTPA) by ITLC and SEC-HPLC at EOS indicated the formation of Compound H (463 μL, 0.713 mg/mL, 0.058 mCi/mg specific activity, 99.5% radiochemical purity, and 98.800 chemical purity).

### Example 5. In Vitro Binding of [¹⁷⁷Lu]-Compound C-Anti-CLDN 18.2 Conjugates

A study was conducted to evaluate the receptor binding affinity of [¹⁷⁷Lu]-Compound C-anti-CLDN 18.2 conjugates, i.e., Compounds F and G, with the MiaPaCa2 K18.2 cell line; and the binding affinity of Compound F with the PATU8988S cell line. This study followed the procedures described below.

The purpose of this assay was to ensure that the conjugation and radiolabeling maintained the binding characteristics of the native antibody in Claudin 18.2 expressing cell lines. Twenty-four hours prior to the start of the experiment, 3×10⁵ MiaPaCa2 K18.2 and PATU8988S cells were seeded in 48-well microplates in 500 μL supplemented medium. The radioimmunoconjugates, Compounds F and G, were diluted with binding buffer (PBS+0.5% BSA) to a range of concentrations from 0.4 nM to 200 nM to achieve final assay concentration of 0.2 nM to 100 nM. At the start of the assay, the media was aspirated, discarded and 500 μL of serum-free media was added to each well. The plates were incubated at 37° C. for 1 hour, following which the cells were washed and 100 μL of binding buffer (total binding)(TB) or 20 pM cold antibody (non-specific binding)(NSB) was added to designated wells. Plates were incubated at 4° C. for 1 hour with mild shaking. Following the blocking step, 100 μL of radioimmunoconjugate was added to each well. The plates were then incubated at 4° C. for 2 hours. Following incubation, the cells were washed twice with PBS and were then lysed with 1% Triton-X-100. The lysates were transferred to gamma counting tubes and run along with radioimmunoconjugate standards on the Wizard 1470 gamma counter to determine the radioactivity content (in counts per minute)(CPM) for each lysate. The remaining lysate from each well (25 μL) was used for analyzing the protein content of each lysate using a standard protein quantification assay.

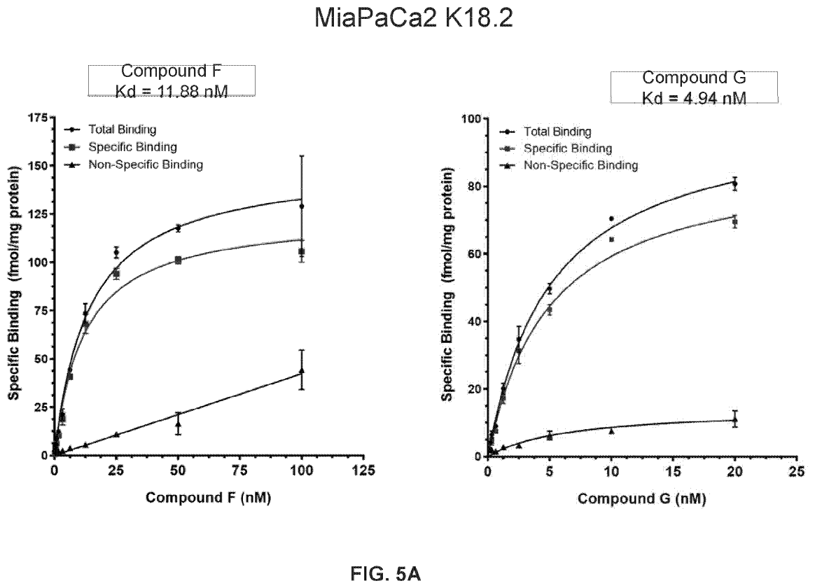

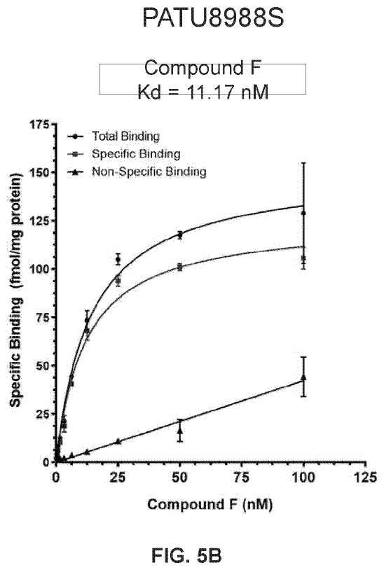

Total, specific, and non-specific binding values (fmol/mg) were plotted against conjugate concentration as shown in FIGS. 5A-5B. The Kd, and Bmax were derived by curve fitting of the specific binding data to a single-site hyperbola model (Graph Pad Prism Software, version 9).

It was observed that the two CLDN 18.2 radioimmunoconjugates, i.e., Compounds F and G, exhibited high binding affinities of 11.88 nM and 4.94 nM, respectively, with the MiaPaCa2 K18.2 cell line; and Compound F exhibited high binding affinities of 11.17 nM with the PATU8988S cell line. The binding data indicates very similar binding affinity for Compound F in Claudin 18.2 exogenously as well as endogenously expressing cell lines.

### Example 6. Internalization Evaluation of [¹⁷⁷Lu]-Compound C-Anti-CLDN 18.2 Conjugate

This internalization assay was designed to determine the degree of cell retention of radiolabeled—linker antibody derivatives. The assay relies on the inherent ability of the Claudin 18.2 receptor to internalize when bound to antibody and the ability to track radiolabeled compounds. Here, a constant amount of radioimmunoconjugate is incubated with an Claudin 18.2 expressing cell line for a fixed duration of time and residualization is determined by calculating the amount of internalized radioactivity as a percentage of the total cell-associated activity.

A study was conducted to evaluate the internalization of a [¹⁷⁷Lu]-Compound C-anti-CLDN 18.2 conjugate, i.e., Compound F, with the MiaPaCa2 K18.2 and PATU8988S cell lines following the protocol described below.

Claudin 18.2 expressing cell lines MiaPaCa2 K18.2 and PATU8988s were plated in 24-well plates at a concentration of 3×10⁵ cells/well in complete medium (DMEM). Next day, the cells were changed to serum-free DMEM and incubated for 1 hour at 37° C. Media was decanted and plates were washed once with sterile PBS. Compound F was diluted in serum free DMEM to a concentration of 22 nM and 500 μL of radioimmunoconjugate was loaded into each well and incubated for 2 hours at 37° C. After incubation, plates were immediately placed on ice and medium was discarded into pre-labeled (non-bound) gamma counting tubes. Cells were washed once with sterile PBS, gently shaken, and decanted into the (non-bound labeled) gamma tubes. Mild acid wash buffer (pH 4.6, 500 μL) was added into 0-hour time point wells. Plates were incubated at 4° C. for 15 minutes and buffer was collected into pre-labeled (membrane-bound) gamma counting tubes. Cells were then lysed with 300 μL 1% Triton X-100 for 30 minutes at room temperature with gentle shaking. 250 μL of the cell lysate was transferred into gamma counting tubes and counted for 10 minutes. For 2-hour and 24-hour incubation, 1 ml of warmed serum-free DMEM media was added to 2-hour and 24-hour time point plate wells and plates were further incubated at 37° C. for 2 and 24 hours respectively. Following the prescribed incubation times, plates were placed on ice and processed in the following manner—media was decanted and collected into pre-labeled (efflux) gamma tubes. Plates were then washed once with 1 ml cold PBS and added into efflux tubes. Strong acid wash buffer (pH 2.5, 500 μL) was added to all wells and plates were incubated for 5 minutes on ice. The acid wash fraction was then collected into pre-labeled (recycled) gamma tubes. Cells were lysed with 300 μL 1% Triton X-100 for 30 minutes at room temperature. 250 μL of the cell lysate was transferred into pre-labeled (retained) gamma counting tubes and counted for 10 minutes. 25 μL of the cell lysate fraction was transferred to a 96-well plate for protein quantification (Pierce BCA Protein Assay). Percent residualization (FIG. 3) was determined as CPM (lysate) or CPM (efflux+recycled+lysate). This in vitro residualization experiment demonstrated very similar cellular internalization and retention rates for Compound F in both the cell lines.

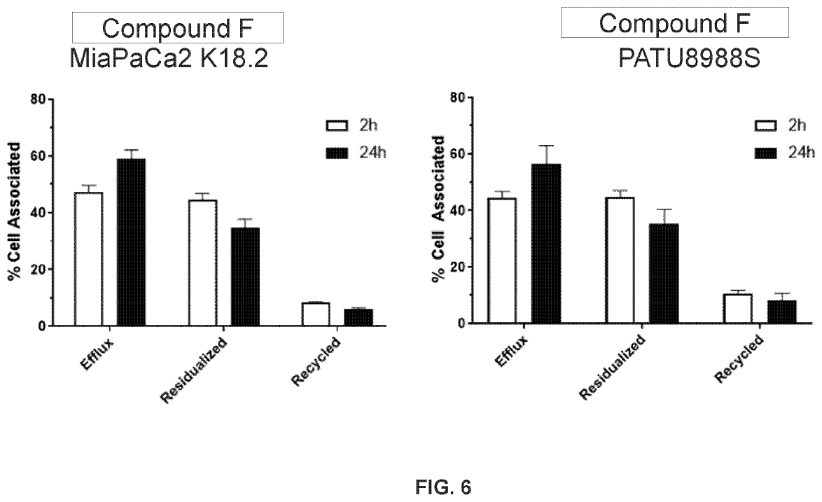

The results from the internalization study are shown in FIG. 6. It was observed that Compound F had about 35% internalized at 24 hr post-incubation with no significant difference between the two cell lines.

### Example 7. In Vivo Biodistribution of [¹⁷⁷Lu]-Compound C-Anti-CLDN 18.2 Conjugates in MiaPaCa2 K18.2 Model

A MiaPaCa2 K18.2 cell line xenograft mouse model was used to assess the in vivo biodistribution of [¹⁷⁷Lu]-DOTA-anti-CLDN 18.2 conjugates following the below protocol.

Tumor inoculations: MiaPaCa2 K18.2 cells were washed with PBS and then detached with 0.25% trypsin EDTA. Washed cells were resuspended in a 50:50 mixture of PBS and Matrigel (BD, Oakville ON) at a concentration of 70×10⁶ cells/mL and 100 μL of the mixture (approximately 7×10⁶ cells) was injected subcutaneously into the right rear-flank of individual mice. Tumor xenografts are established in 5- to 7-week-old female Balb/c NCI Athymic NCr-nu/nu mice (Charles River Laboratories). Xenografts were allowed to grow for a minimum of 7 days to an initial volume of 150-200 mm³ before initiation of treatment.

Biodistribution studies were performed using MiaPaCa2 K18.2 xenograft bearing mice generated as described above. Day 7 post tumor inoculation animals were injected intravenously via the lateral tail vein with 200 μL of radioimmunoconjugates, Compounds F and G, containing approximately 0.37-0.74 MBq of Lu-177 (approximately 1-2 μg of antibody). At specified timepoints (4 h, 24 h, 96 h, and 168 h) post injection, 3 animals per timepoint were anesthetized with isoflurane, exsanguinated via cardiac puncture then euthanized for blood and different organ collection by dissection. Tumor and organs were rinsed with PBS of any residual blood, blotted dry and collected into pre-weighed gamma counting tubes to determine levels of radioactivity in the blood, heart, intestines, esophagus, pancreas, stomach, kidneys, liver, lungs, spleen, tumor, urine, and tail. Radiation counts per minute contained in tissue samples were measured using a gamma counter then converted to decay corrected μCi of activity using a calibration standard. Activity measurements and sample weights were used to calculate the percent of injected dose per gram of tissue weight (% ID/g).

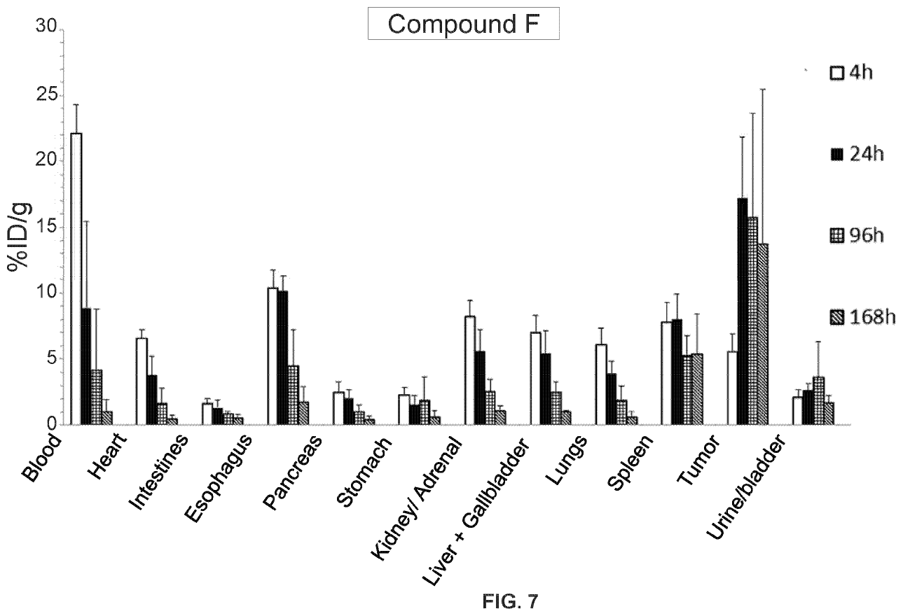

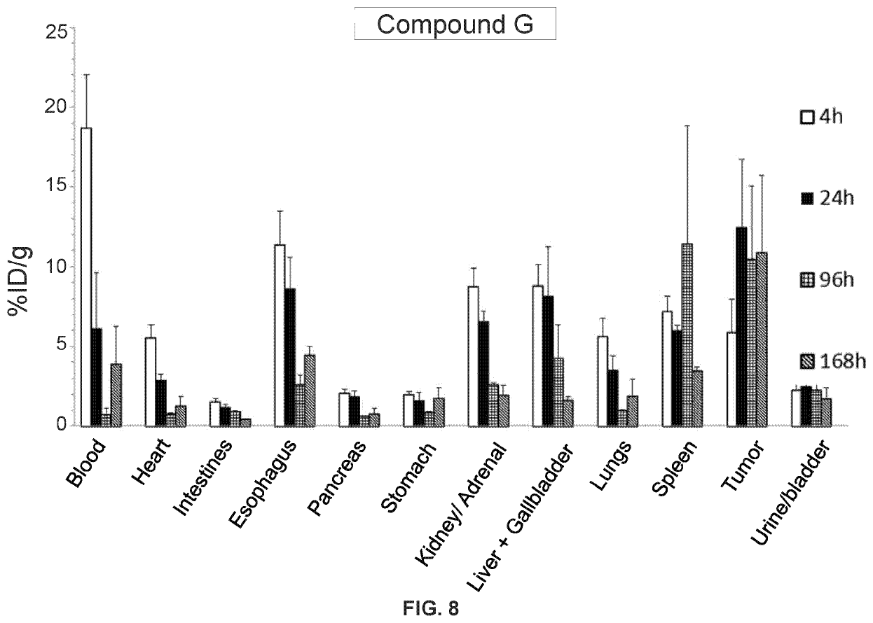

Results were expressed as the percentage injected dose per gram of tissue (% ID/g) and are depicted in FIGS. 7-8. Biodistribution study of Compound F showed highest uptake of 17% (based on % ID/g) in tumors while minimal uptake was observed in normal organs. Conjugate B showed highest uptake of 12% in tumors with slightly higher uptake of 8-11% in liver and spleen as well.

### Example 8. In Vivo Efficacy of [²²⁵Ac]-Compound C-Anti-CLDN 18.2 Conjugate in MiaPaCa2 K18.2 Model

A study was designed to evaluate the efficacy for different doses of actinium-225 labeled radioimmunoconjugate, Compound H, as compared to the cold-antibody D03 clone and vehicle control.

The efficacy study was carried out using the Claudin 18.2 overexpressing pancreatic cancer cell line, MiaPaCa2 K18.2. Tumor xenografts were established as mentioned above. On day 7 post-inoculation groups of tumor-bearing animals (n=5) were injected intravenously via the lateral tail vein with 200 μL of test article, Compound H, which was dosed at 50-400 nanocuries (nCi) of activity formulated in 20 mM sodium citrate pH 5.5, 0.82% NaCl, and 0.01% Tween-80 buffer. As a control, non-radiolabeled, non-conjugated antibody D03 (Antibody control) was administered at a protein mass equivalent corresponding to the highest radioactivity dose of the actinium-225 radioimmunoconjugates tested in the study. Tumor measurements were taken 2-3 times per week for 28 days with vernier calipers in two dimensions. Tumor length was defined as the longest dimension, width was measured perpendicular to the tumor length. At the same time animals were weighed. Overall body condition and general behavior were assessed daily. Tumor volume (mm³) was calculated from caliper measurements as an ellipsoid: Tumor growth was expressed as relative tumor volume (RTV) which is tumor volume measured on day X divided by the tumor volume measured on the day of dosing.

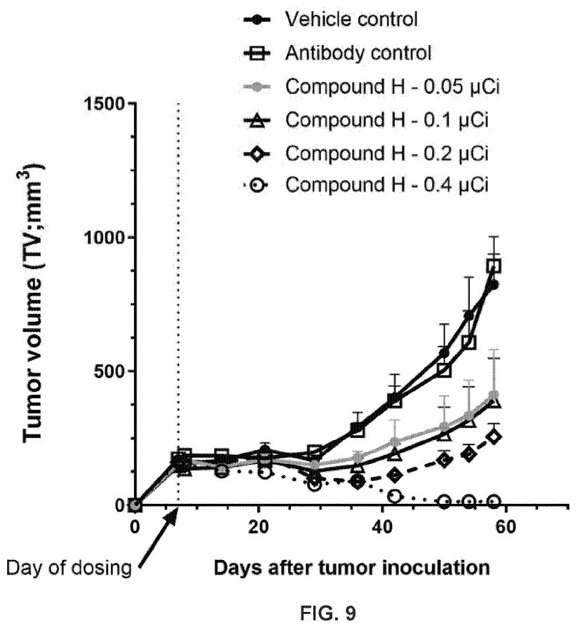

Results from the efficacy study are shown in FIG. 9. A dose-dependent reduction in tumor volume was observed with a single administration of Compound H in comparison to controls. Tumor regression was observed at the highest dose of Compound H (0.4 μCi), while significant tumor growth inhibition (73%) was observed at the 0.2 μCi dose in comparison to control groups at endpoint (day 58).

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A compound comprising the structure of Formula I, or a pharmaceutically acceptable salt thereof: 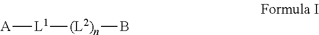
wherein
A is a chelating moiety or a metal complex thereof,
B is a targeting moiety that is capable of binding to Claudin 18.2 (CLDN18.2) or a fragment thereof;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II: 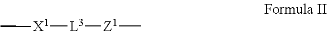
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀ alkyl or optionally substituted C₁-C₅₀ heteroalkyl; and
Z¹ is —CH₂—#, —C(O)—#, —C(S)—#, —OC(O)—#, —C(O)O—#, —NR²C(O)—#, —C(O)NR²—#, or —NR²—#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

2. The compound of claim 1, wherein said chelating moiety is selected from the group consisting of DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaoxacyclohexadecane-1,4,7,10,13,16-hexaacetic acid), PEPA (1,4,7,10,13-pentaazacyclopentadecane-N,N′,N″,N′″,N″″-pentaacetic acid), H₄ octapa (N,N′-bis(6-carboxy-2-pyridylmethyl)-ethylenediamine-N,N′-diacetic acid), H₂ dedpa (1,2-[[6-(carboxy)-pyridin-2-yl]-methylamino]ethane), H₆ phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″,N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

3. The compound of claim 2, wherein the compound is represented by: 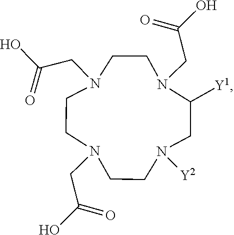
wherein Y¹ is —CH₂OCH₂(L²)ₙ-B, C═O(L²)ₙ-B, or C═S(L²)ₙ-B and Y² is —CH₂CO₂H; or
wherein Y¹ is H and Y² is L¹-(L²)ₙ-B.

4. The compound of any one of claims 1-3, wherein L¹ is 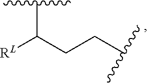
wherein R^{L} is hydrogen or —CO₂H.

5. The compound of any one of claims 1-4,
wherein the metal complex comprises a metal selected from the group consisting of Bi, Pb, Y, Mn, Cr, Fe, Co, Zn, Ni, Tc, In, Ga, Cu, Re, a lanthanide, and an actinide; or
wherein the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹ At, ²¹²Pb, ²¹²Bi, ²¹³Bi, ²²³Ra, ²²⁵Ac, ²²⁷Th, and ²²⁹Th.

6. The compound of any one of claims 3-5, wherein Y¹ is H.

7. The compound of any one of claims 1-6 wherein X¹ is —C(O)NR¹—* or —NR¹C(O)—*, “*” indicating the attachment point to L³, and R¹ is H.

8. The compound of any one of claims 1-7, wherein Z¹ is —CH₂—.

9. The compound of any one of claims 1-8, wherein n is 1, and L³ comprises (CH₂CH₂O)₂₋₂₀.

10. The compound of any one of claims 1-8, wherein n is 1, and L³ is (CH₂CH₂O)ₘ(CH₂)_{w}, wherein m and w are each independently an integer between 0 and 10 (inclusive), and at least one of m and w is not 0.

11. The compound of claim 1, wherein A-L- comprises one of the following structures, or a metal complex thereof:

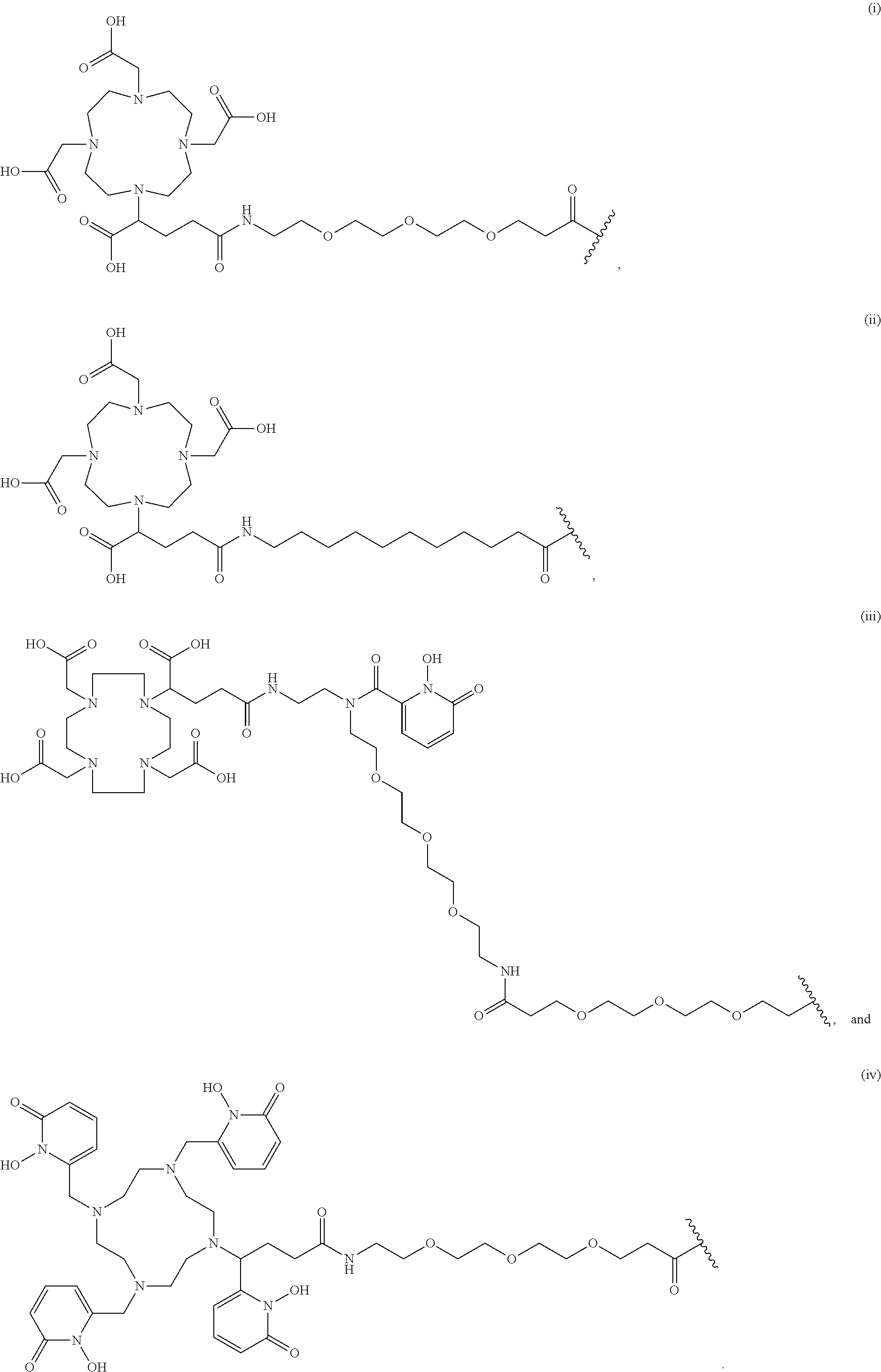 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O ; *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

12. The compound of claim 1, wherein the compound comprises the following structure, or a metal complex thereof:

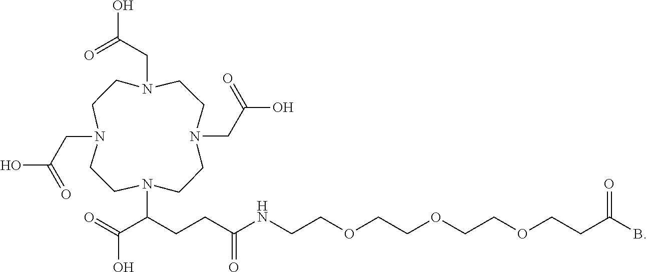

13. The compound of any one of claims 1-12, wherein A is a metal complex of a chelating moiety, and the metal complex comprises a radionuclide.

14. The compound of claim 13, wherein the radionuclide is ⁶⁸Ga, ¹¹¹In, ¹⁷⁷Lu, or ²²⁵Ac.

15. The compound of claim 13, wherein the radionuclide is ²²⁵Ac.

16. The compound of claim 13, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

17. The compound of claim 16, wherein the alpha emitter is ²²⁵Ac or a progeny thereof.

18. The compound of any one of claims 1-17, wherein the targeting moiety comprises an antibody or antigen-binding fragment thereof that is capable of binding to CLDN18.2 or a fragment thereof.

19. The compound of claim 18, wherein said antibody or antigen-binding fragment thereof comprises:
(a)(i) a heavy chain variable domain (VH) comprising complementarity-determining regions:
CDR-H1 comprising the amino acid sequence of SEQ ID NO: 2 or a sequence differing in 1 or 2 amino acids therefrom,
CDR-H2 comprising the amino acid sequence of SEQ ID NO: 3 or a sequence differing in 1 or 2 amino acids therefrom, and
CDR-H3 comprising the amino acid sequence of SEQ ID NO: 4 or a sequence differing in 1 or 2 amino acids therefrom; and
(ii) a light chain variable domain (VL) comprising complementarity-determining regions:
CDR-L1 comprising the amino acid sequence of SEQ ID NO: 6 or a sequence differing in 1 or 2 amino acids therefrom,
CDR-L2 comprising the amino acid sequence of SEQ ID NO: 7 or a sequence differing in 1 or 2 amino acids therefrom, and
CDR-L3 comprising the amino acid sequence of SEQ ID NO: 8 or a sequence differing in 1 or 2 amino acids therefrom;
(b)(i) a VH comprising complementarity-determining regions:
CDR-H1 comprising the amino acid sequence of SEQ ID NO: 10 or a sequence differing in 1 or 2 amino acids therefrom,
CDR-H2 comprising the amino acid sequence of SEQ ID NO: 11 or a sequence differing in 1 or 2 amino acids therefrom, and
CDR-H3 comprising the amino acid sequence of SEQ ID NO: 12 or a sequence differing in 1 or 2 amino acids therefrom; and
(ii) a VL comprising complementarity-determining regions:
CDR-L1 comprising the amino acid sequence of SEQ ID NO: 14 or a sequence differing in 1 or 2 amino acids therefrom,
CDR-L2 comprising the amino acid sequence of SEQ ID NO: 15 or a sequence differing in 1 or 2 amino acids therefrom, and
CDR-L3 comprising the amino acid sequence of SEQ ID NO: 16 or a sequence differing in 1 or 2 amino acids therefrom;
(c)(i) a VH comprising complementarity-determining regions:
CDR-H1 comprising the amino acid sequence of SEQ ID NO: 18 or a sequence differing in 1 or 2 amino acids therefrom,
CDR-H2 comprising the amino acid sequence of SEQ ID NO: 19 or a sequence differing in 1 or 2 amino acids therefrom, and
CDR-H3 comprising the amino acid sequence of SEQ ID NO: 20 or a sequence differing in 1 or 2 amino acids therefrom; and
(ii) a VL comprising complementarity-determining regions:
CDR-L1 comprising the amino acid sequence of SEQ ID NO: 22 or a sequence differing in 1 or 2 amino acids therefrom,
CDR-L2 comprising the amino acid sequence of SEQ ID NO: 23 or a sequence differing in 1 or 2 amino acids therefrom, and
CDR-L3 comprising the amino acid sequence of SEQ ID NO: 24 or a sequence differing in 1 or 2 amino acids therefrom;
(d)(i) a VH comprising complementarity-determining regions:
CDR-H1 comprising the amino acid sequence of SEQ ID NO: 26 or a sequence differing in 1 or 2 amino acids therefrom,
CDR-H2 comprising the amino acid sequence of SEQ ID NO: 27 or a sequence differing in 1 or 2 amino acids therefrom, and
CDR-H3 comprising the amino acid sequence of SEQ ID NO: 28 or a sequence differing in 1 or 2 amino acids therefrom; and
(ii) a VL comprising complementarity-determining regions:
CDR-L1 comprising the amino acid sequence of SEQ ID NO: 30 or a sequence differing in 1 or 2 amino acids therefrom,
CDR-L2 comprising the amino acid sequence of SEQ ID NO: 31 or a sequence differing in 1 or 2 amino acids therefrom, and
CDR-L3 comprising the amino acid sequence of SEQ ID NO: 32 or a sequence differing in 1 or 2 amino acids therefrom;
(e)(i) a VH comprising complementarity-determining regions:
CDR-H1 comprising the amino acid sequence of SEQ ID NO: 34 or a sequence differing in 1 or 2 amino acids therefrom,
CDR-H2 comprising the amino acid sequence of SEQ ID NO: 35 or a sequence differing in 1 or 2 amino acids therefrom, and
CDR-H3 comprising the amino acid sequence of SEQ ID NO: 36 or a sequence differing in 1 or 2 amino acids therefrom; and
(ii) a VL comprising complementarity-determining regions:
CDR-L1 comprising the amino acid sequence of SEQ ID NO: 38 or a sequence differing in 1 or 2 amino acids therefrom,
CDR-L2 comprising the amino acid sequence of SEQ ID NO: 39 or a sequence differing in 1 or 2 amino acids therefrom, and
CDR-L3 comprising the amino acid sequence of SEQ ID NO: 40 or a sequence differing in 1 or 2 amino acids therefrom;
(f)(i) a VH comprising complementarity-determining regions:
CDR-H1 comprising the amino acid sequence of SEQ ID NO: 42 or a sequence differing in 1 or 2 amino acids therefrom,
CDR-H2 comprising the amino acid sequence of SEQ ID NO: 43 or a sequence differing in 1 or 2 amino acids therefrom, and
CDR-H3 comprising the amino acid sequence of SEQ ID NO: 44 or a sequence differing in 1 or 2 amino acids therefrom; and
(ii) a VL comprising complementarity-determining regions:
CDR-L1 comprising the amino acid sequence of SEQ ID NO: 46 or a sequence differing in 1 or 2 amino acids therefrom,
CDR-L2 comprising the amino acid sequence of SEQ ID NO: 47 or a sequence differing in 1 or 2 amino acids therefrom, and
CDR-L3 comprising the amino acid sequence of SEQ ID NO: 48 or a sequence differing in 1 or 2 amino acids therefrom;
(g)(i) a VH comprising complementarity-determining regions:
CDR-H1 comprising the amino acid sequence of SEQ ID NO: 50 or a sequence differing in 1 or 2 amino acids therefrom,
CDR-H2 comprising the amino acid sequence of SEQ ID NO: 51 or a sequence differing in 1 or 2 amino acids therefrom, and
CDR-H3 comprising the amino acid sequence of SEQ ID NO: 52 or a sequence differing in 1 or 2 amino acids therefrom; and
(ii) a VL comprising complementarity-determining regions:
CDR-L1 comprising the amino acid sequence of SEQ ID NO: 54 or a sequence differing in 1 or 2 amino acids therefrom,
CDR-L2 comprising the amino acid sequence of SEQ ID NO: 55 or a sequence differing in 1 or 2 amino acids therefrom, and
CDR-L3 comprising the amino acid sequence of SEQ ID NO: 56 or a sequence differing in 1 or 2 amino acids therefrom;
(h)(i) a VH comprising complementarity-determining regions:
CDR-H1 comprising the amino acid sequence of SEQ ID NO: 58 or a sequence differing in 1 or 2 amino acids therefrom,
CDR-H2 comprising the amino acid sequence of SEQ ID NO: 59 or a sequence differing in 1 or 2 amino acids therefrom, and
CDR-H3 comprising the amino acid sequence of SEQ ID NO: 60 or a sequence differing in 1 or 2 amino acids therefrom; and
(ii) a VL comprising complementarity-determining regions:
CDR-L1 comprising the amino acid sequence of SEQ ID NO: 62 or a sequence differing in 1 or 2 amino acids therefrom,
CDR-L2 comprising the amino acid sequence of SEQ ID NO: 63 or a sequence differing in 1 or 2 amino acids therefrom, and
CDR-L3 comprising the amino acid sequence of SEQ ID NO: 64 or a sequence differing in 1 or 2 amino acids therefrom;
(i)(i) a VH comprising complementarity-determining regions:
CDR-H1 comprising the amino acid sequence of SEQ ID NO: 66 or a sequence differing in 1 or 2 amino acids therefrom,
CDR-H2 comprising the amino acid sequence of SEQ ID NO: 67 or a sequence differing in 1 or 2 amino acids therefrom, and
CDR-H3 comprising the amino acid sequence of SEQ ID NO: 68 or a sequence differing in 1 or 2 amino acids therefrom; and
(ii) a VL comprising complementarity-determining regions:
CDR-L1 comprising the amino acid sequence of SEQ ID NO: 70 or a sequence differing in 1 or 2 amino acids therefrom,
CDR-L2 comprising the amino acid sequence of SEQ ID NO: 71 or a sequence differing in 1 or 2 amino acids therefrom, and
CDR-L3 comprising the amino acid sequence of SEQ ID NO: 72 or a sequence differing in 1 or 2 amino acids therefrom;
(j)(i) a VH comprising complementarity-determining regions:
CDR-H1 comprising the amino acid sequence of SEQ ID NO: 74 or a sequence differing in 1 or 2 amino acids therefrom,
CDR-H2 comprising the amino acid sequence of SEQ ID NO: 75 or a sequence differing in 1 or 2 amino acids therefrom, and
CDR-H3 comprising the amino acid sequence of SEQ ID NO: 76 or a sequence differing in 1 or 2 amino acids therefrom; and
(ii) a VL comprising complementarity-determining regions:
CDR-L1 comprising the amino acid sequence of SEQ ID NO: 78 or a sequence differing in 1 or 2 amino acids therefrom,
CDR-L2 comprising the amino acid sequence of SEQ ID NO: 79 or a sequence differing in 1 or 2 amino acids therefrom, and
CDR-L3 comprising the amino acid sequence of SEQ ID NO: 80 or a sequence differing in 1 or 2 amino acids therefrom;
(k)(i) a VH comprising complementarity-determining regions:
CDR-H1 comprising the amino acid sequence of SEQ ID NO: 82 or a sequence differing in 1 or 2 amino acids therefrom,
CDR-H2 comprising the amino acid sequence of SEQ ID NO: 83 or a sequence differing in 1 or 2 amino acids therefrom, and
CDR-H3 comprising the amino acid sequence of SEQ ID NO: 84 or a sequence differing in 1 or 2 amino acids therefrom; and
(ii) a VL comprising complementarity-determining regions:
CDR-L1 comprising the amino acid sequence of SEQ ID NO: 86 or a sequence differing in 1 or 2 amino acids therefrom,
CDR-L2 comprising the amino acid sequence of SEQ ID NO: 87 or a sequence differing in 1 or 2 amino acids therefrom, and
CDR-L3 comprising the amino acid sequence of SEQ ID NO: 88 or a sequence differing in 1 or 2 amino acids therefrom;
(1)(i) a VH comprising complementarity-determining regions:
CDR-H1 comprising the amino acid sequence of SEQ ID NO: 90 or a sequence differing in 1 or 2 amino acids therefrom,
CDR-H2 comprising the amino acid sequence of SEQ ID NO: 91 or a sequence differing in 1 or 2 amino acids therefrom, and
CDR-H3 comprising the amino acid sequence of SEQ ID NO: 92 or a sequence differing in 1 or 2 amino acids therefrom; and
(ii) a VL comprising complementarity-determining regions:
CDR-L1 comprising the amino acid sequence of SEQ ID NO: 94 or a sequence differing in 1 or 2 amino acids therefrom,
CDR-L2 comprising the amino acid sequence of SEQ ID NO: 95 or a sequence differing in 1 or 2 amino acids therefrom, and
CDR-L3 comprising the amino acid sequence of SEQ ID NO: 96 or a sequence differing in 1 or 2 amino acids therefrom;
(m)(i) a VH comprising complementarity-determining regions:
CDR-H1 comprising the amino acid sequence of SEQ ID NO: 98 or a sequence differing in 1 or 2 amino acids therefrom,
CDR-H2 comprising the amino acid sequence of SEQ ID NO: 99 or a sequence differing in 1 or 2 amino acids therefrom, and
CDR-H3 comprising the amino acid sequence of SEQ ID NO: 100 or a sequence differing in 1 or 2 amino acids therefrom; and
(ii) a VL comprising complementarity-determining regions:
CDR-L1 comprising the amino acid sequence of SEQ ID NO: 102 or a sequence differing in 1 or 2 amino acids therefrom,
CDR-L2 comprising the amino acid sequence of SEQ ID NO: 103 or a sequence differing in 1 or 2 amino acids therefrom, and
CDR-L3 comprising the amino acid sequence of SEQ ID NO: 104 or a sequence differing in 1 or 2 amino acids therefrom; or
(n)(i) a VH comprising complementarity-determining regions:
CDR-H1 comprising the amino acid sequence of SEQ ID NO: 106 or a sequence differing in 1 or 2 amino acids therefrom,
CDR-H2 comprising the amino acid sequence of SEQ ID NO: 107 or a sequence differing in 1 or 2 amino acids therefrom, and
CDR-H3 comprising the amino acid sequence of SEQ ID NO: 108 or a sequence differing in 1 or 2 amino acids therefrom; and
(ii) a VL comprising complementarity-determining regions:
CDR-L1 comprising the amino acid sequence of SEQ ID NO: 110 or a sequence differing in 1 or 2 amino acids therefrom,
CDR-L2 comprising the amino acid sequence of SEQ ID NO: 111 or a sequence differing in 1 or 2 amino acids therefrom, and
CDR-L3 comprising the amino acid sequence of SEQ ID NO: 112 or a sequence differing in 1 or 2 amino acids therefrom.

20. The compound of claim 19, wherein the CDR-H1, CDR-H2, CDR-H3, CDR-L1, CDR-L2, and CDR-L3 comprise amino acid sequences that collectively differ by no more than two amino acid residues from the sequences of:
(a) SEQ ID NO: 2, SEQ ID NO: 3, SEQ ID NO: 4, SEQ ID NO: 6, SEQ ID NO: 7, and SEQ ID NO: 8, respectively;
(b) SEQ ID NO: 10, SEQ ID NO: 11, SEQ ID NO: 12, SEQ ID NO: 14, SEQ ID NO:15, and SEQ ID NO: 16, respectively;
(c) SEQ ID NO: 18, SEQ ID NO: 19, SEQ ID NO: 20, SEQ ID NO: 22, SEQ ID NO:23, SEQ ID NO: 24, respectively;
(d) SEQ ID NO: 26, SEQ ID NO: 27, SEQ ID NO: 28, SEQ ID NO: 30, SEQ ID NO: 31, SEQ ID NO: 32, respectively;
(e) SEQ ID NO: 34, SEQ ID NO: 35, SEQ ID NO: 36, SEQ ID NO: 38, SEQ ID NO: 39, SEQ ID NO: 40, respectively;
(f) SEQ ID NO: 42, SEQ ID NO: 43, SEQ ID NO: 44, SEQ ID NO: 46, SEQ ID NO: 47, SEQ ID NO: 48, respectively;
(g) SEQ ID NO: 50, SEQ ID NO: 51, SEQ ID NO: 52, SEQ ID NO: 54, SEQ ID NO: 55, SEQ ID NO: 56, respectively;
(h) SEQ ID NO: 58, SEQ ID NO: 59, SEQ ID NO: 60, SEQ ID NO: 62, SEQ ID NO: 63, SEQ ID NO: 64, respectively;
(i) SEQ ID NO: 66, SEQ ID NO: 67, SEQ ID NO: 68, SEQ ID NO: 70, SEQ ID NO:71, SEQ ID NO: 72, respectively;
(j) SEQ ID NO: 74, SEQ ID NO: 75, SEQ ID NO: 76, SEQ ID NO: 78, SEQ ID NO: 79, SEQ ID NO: 80, respectively;
(k) SEQ ID NO: 82, SEQ ID NO: 83, SEQ ID NO: 84, SEQ ID NO: 86, SEQ ID NO: 87, SEQ ID NO: 88, respectively;
(l) SEQ ID NO: 90, SEQ ID NO: 91, SEQ ID NO: 92, SEQ ID NO: 94, SEQ ID NO: 95, SEQ ID NO: 96, respectively;
(m) SEQ ID NO: 98, SEQ ID NO: 99, SEQ ID NO: 100, SEQ ID NO: 102, SEQ ID NO: 103, SEQ ID NO: 104, respectively; or
(n) SEQ ID NO: 106, SEQ ID NO: 107, SEQ ID NO: 108, SEQ ID NO: 110, SEQ ID NO: 111, SEQ ID NO: 112, respectively.

21. The compound of claim 20, wherein said antibody or antigen-binding fragment thereof comprises:
(a)(i) a VH comprising complementarity-determining regions:
CDR-H1 comprising the amino acid sequence of SEQ ID NO: 2,
CDR-H2 comprising the amino acid sequence of SEQ ID NO: 3, and
CDR-H3 comprising the amino acid sequence of SEQ ID NO: 4; and
(ii) a VL comprising complementarity-determining regions:
CDR-L1 comprising the amino acid sequence of SEQ ID NO: 6,
CDR-L2 comprising the amino acid sequence of SEQ ID NO: 7, and
CDR-L3 comprising the amino acid sequence of SEQ ID NO: 8;
(b)(i) a VH comprising complementarity-determining regions:
CDR-H1 comprising the amino acid sequence of SEQ ID NO: 10,
CDR-H2 comprising the amino acid sequence of SEQ ID NO: 11, and
CDR-H3 comprising the amino acid sequence of SEQ ID NO: 12; and
(ii) a VL comprising complementarity-determining regions:
CDR-L1 comprising the amino acid sequence of SEQ ID NO: 14,
CDR-L2 comprising the amino acid sequence of SEQ ID NO: 15, and
CDR-L3 comprising the amino acid sequence of SEQ ID NO: 16;
(c)(i) a VH comprising complementarity-determining regions:
CDR-H1 comprising the amino acid sequence of SEQ ID NO: 18,
CDR-H2 comprising the amino acid sequence of SEQ ID NO: 19, and
CDR-H3 comprising the amino acid sequence of SEQ ID NO: 20; and
(ii) a VL comprising complementarity-determining regions:
CDR-L1 comprising the amino acid sequence of SEQ ID NO: 22,
CDR-L2 comprising the amino acid sequence of SEQ ID NO: 23, and
CDR-L3 comprising the amino acid sequence of SEQ ID NO: 24;
(d)(i) a VH comprising complementarity-determining regions:
CDR-H1 comprising the amino acid sequence of SEQ ID NO: 26,
CDR-H2 comprising the amino acid sequence of SEQ ID NO: 27, and
CDR-H3 comprising the amino acid sequence of SEQ ID NO: 28; and
(ii) a VL comprising complementarity-determining regions:
CDR-L1 comprising the amino acid sequence of SEQ ID NO: 30,
CDR-L2 comprising the amino acid sequence of SEQ ID NO: 31, and
CDR-L3 comprising the amino acid sequence of SEQ ID NO: 32;
(e)(i) a VH comprising complementarity-determining regions:
CDR-H1 comprising the amino acid sequence of SEQ ID NO: 34,
CDR-H2 comprising the amino acid sequence of SEQ ID NO: 35, and
CDR-H3 comprising the amino acid sequence of SEQ ID NO: 36; and
(ii) a VL comprising complementarity-determining regions:
CDR-L1 comprising the amino acid sequence of SEQ ID NO: 38,
CDR-L2 comprising the amino acid sequence of SEQ ID NO: 39, and
CDR-L3 comprising the amino acid sequence of SEQ ID NO: 40;
(f)(i) a VH comprising complementarity-determining regions:
CDR-H1 comprising the amino acid sequence of SEQ ID NO: 42,
CDR-H2 comprising the amino acid sequence of SEQ ID NO: 43, and
CDR-H3 comprising the amino acid sequence of SEQ ID NO: 44; and
(ii) a VL comprising complementarity-determining regions:
CDR-L1 comprising the amino acid sequence of SEQ ID NO: 46,
CDR-L2 comprising the amino acid sequence of SEQ ID NO: 47, and
CDR-L3 comprising the amino acid sequence of SEQ ID NO: 48;
(g)(i) a VH comprising complementarity-determining regions:
CDR-H1 comprising the amino acid sequence of SEQ ID NO: 50,
CDR-H2 comprising the amino acid sequence of SEQ ID NO: 51, and
CDR-H3 comprising the amino acid sequence of SEQ ID NO: 52; and
(ii) a VL comprising complementarity-determining regions:
CDR-L1 comprising the amino acid sequence of SEQ ID NO: 54,
CDR-L2 comprising the amino acid sequence of SEQ ID NO: 55, and
CDR-L3 comprising the amino acid sequence of SEQ ID NO: 56;
(h)(i) a VH comprising complementarity-determining regions:
CDR-H1 comprising the amino acid sequence of SEQ ID NO: 58,
CDR-H2 comprising the amino acid sequence of SEQ ID NO: 59, and
CDR-H3 comprising the amino acid sequence of SEQ ID NO: 60; and
(ii) a VL comprising complementarity-determining regions:
CDR-L1 comprising the amino acid sequence of SEQ ID NO: 62,
CDR-L2 comprising the amino acid sequence of SEQ ID NO: 63, and
CDR-L3 comprising the amino acid sequence of SEQ ID NO: 64;
(i)(i) a VH comprising complementarity-determining regions:
CDR-H1 comprising the amino acid sequence of SEQ ID NO: 66,
CDR-H2 comprising the amino acid sequence of SEQ ID NO: 67, and
CDR-H3 comprising the amino acid sequence of SEQ ID NO: 68; and
(ii) a VL comprising complementarity-determining regions:
CDR-L1 comprising the amino acid sequence of SEQ ID NO: 70,
CDR-L2 comprising the amino acid sequence of SEQ ID NO: 71, and
CDR-L3 comprising the amino acid sequence of SEQ ID NO: 72;
(j)(i) a VH comprising complementarity-determining regions:
CDR-H1 comprising the amino acid sequence of SEQ ID NO: 74,
CDR-H2 comprising the amino acid sequence of SEQ ID NO: 75, and
CDR-H3 comprising the amino acid sequence of SEQ ID NO: 76; and
(ii) a VL comprising complementarity-determining regions:
CDR-L1 comprising the amino acid sequence of SEQ ID NO: 78,
CDR-L2 comprising the amino acid sequence of SEQ ID NO: 79, and
CDR-L3 comprising the amino acid sequence of SEQ ID NO: 80;
(k)(i) a VH comprising complementarity-determining regions:
CDR-H1 comprising the amino acid sequence of SEQ ID NO: 82,
CDR-H2 comprising the amino acid sequence of SEQ ID NO: 83, and
CDR-H3 comprising the amino acid sequence of SEQ ID NO: 84; and
(ii) a VL comprising complementarity-determining regions:
CDR-L1 comprising the amino acid sequence of SEQ ID NO: 86,
CDR-L2 comprising the amino acid sequence of SEQ ID NO: 87, and
CDR-L3 comprising the amino acid sequence of SEQ ID NO: 88;
(l)(i) a VH comprising complementarity-determining regions:
CDR-H1 comprising the amino acid sequence of SEQ ID NO: 90,
CDR-H2 comprising the amino acid sequence of SEQ ID NO: 91, and
CDR-H3 comprising the amino acid sequence of SEQ ID NO: 92; and
(ii) a VL comprising complementarity-determining regions:
CDR-L1 comprising the amino acid sequence of SEQ ID NO: 94,
CDR-L2 comprising the amino acid sequence of SEQ ID NO: 95, and
CDR-L3 comprising the amino acid sequence of SEQ ID NO: 96;
(m)(i) a VH comprising complementarity-determining regions:
CDR-H1 comprising the amino acid sequence of SEQ ID NO: 98,
CDR-H2 comprising the amino acid sequence of SEQ ID NO: 99, and
CDR-H3 comprising the amino acid sequence of SEQ ID NO: 100; and
(ii) a VL comprising complementarity-determining regions:
CDR-L1 comprising the amino acid sequence of SEQ ID NO: 102,
CDR-L2 comprising the amino acid sequence of SEQ ID NO: 103, and
CDR-L3 comprising the amino acid sequence of SEQ ID NO: 104; or
(n)(i) a VH comprising complementarity-determining regions:
CDR-H1 comprising the amino acid sequence of SEQ ID NO: 106,
CDR-H2 comprising the amino acid sequence of SEQ ID NO: 107, and
CDR-H3 comprising the amino acid sequence of SEQ ID NO: 108; and
(ii) a VL comprising complementarity-determining regions:
CDR-L1 comprising the amino acid sequence of SEQ ID NO: 110,
CDR-L2 comprising the amino acid sequence of SEQ ID NO: 111, and
CDR-L3 comprising the amino acid sequence of SEQ ID NO: 112.

22. The compound of claim 21, wherein the antibody or antigen binding fragment thereof comprises:
(i) a VH comprising complementarity-determining regions:
CDR-H1 comprising the amino acid sequence of SEQ ID NO: 2;
CDR-H2 comprising the amino acid sequence of SEQ ID NO: 3; and
CDR-H3 comprising the amino acid sequence of SEQ ID NO: 4; and
(ii) a VL comprising complementarity-determining regions:
CDR-L1 comprising the amino acid sequence of SEQ ID NO: 6;
CDR-L2 comprising the amino acid sequence of SEQ ID NO: 7; and
CDR-L3 comprising the amino acid sequence of SEQ ID NO: 8.

23. The compound of any one of claims 18-22, wherein the antibody or antigen binding fragment thereof comprises:
a VH comprises an amino acid sequence that is at least 85% identical to that of SEQ ID NO: 1; and
a VL comprises an amino acid sequence that is at least 85% identical to that of SEQ ID NO: 5.

24. The compound of claim 23, wherein the antibody or antigen binding fragment thereof comprises:
a VH comprising the amino acid sequence of SEQ ID NO: 1; and
a VL comprising the amino acid sequence of SEQ ID NO: 5.

25. The compound of claim 21, wherein the antibody or antigen binding fragment thereof comprises:
(i) a VH comprising complementarity-determining regions:
CDR-H1 comprising the amino acid sequence of SEQ ID NO: 82;
CDR-H2 comprising the amino acid sequence of SEQ ID NO: 83; and
CDR-H3 comprising the amino acid sequence of SEQ ID NO: 84; and
(ii) a VL comprising complementarity-determining regions:
CDR-L1 comprising the amino acid sequence of SEQ ID NO: 86;
CDR-L2 comprising the amino acid sequence of SEQ ID NO: 87; and
CDR-L3 comprising the amino acid sequence of SEQ ID NO: 88;

26. The compound of any one of claims 19-21 or 25, wherein the antibody or antigen binding fragment thereof comprises:
a VH comprises an amino acid sequence that is at least 85% identical to that of SEQ ID NO: 81; and
a VL comprises an amino acid sequence that is at least 85% identical to that of SEQ ID NO: 85.

27. The compound of claim 26, wherein the antibody or antigen binding fragment thereof comprises:
a VH comprising the amino acid sequence of SEQ ID NO: 81; and
a VL comprising the amino acid sequence of SEQ ID NO: 85.

28. The compound of claim 19, wherein:
(a) the VH comprises an amino acid sequence at least 90%, 95%, 96%, 97%, 98%, or 99% identical to SEQ ID NO: 1 and the VL comprises an amino acid sequence at least 90%, 95%, 96%, 97%, 98%, or 99% identical to SEQ ID NO: 5;
(b) the VH comprises an amino acid sequence at least 90%, 95%, 96%, 97%, 98%, or 99% identical to SEQ ID NO: 9 and the VL comprises an amino acid sequence at least 90%, 95%, 96%, 97%, 98%, or 99% identical to SEQ ID NO: 13;
(c) the VH comprises an amino acid sequence at least 90%, 95%, 96%, 97%, 98%, or 99% identical to SEQ ID NO: 17 and the VL comprises an amino acid sequence at least 90%, 95%, 96%, 97%, 98%, or 99% identical to SEQ ID NO: 21;
(d) the VH comprises an amino acid sequence at least 90%, 95%, 96%, 97%, 98%, or 99% identical to SEQ ID NO: 25 and the VL comprises an amino acid sequence at least 90%, 95%, 96%, 97%, 98%, or 99% identical to SEQ ID NO: 29;
(e) the VH comprises an amino acid sequence at least 90%, 95%, 96%, 97%, 98%, or 99% identical to SEQ ID NO: 33 and the VL comprises an amino acid sequence at least 90%, 95%, 96%, 97%, 98%, or 99% identical to SEQ ID NO: 37;
(f) the VH comprises an amino acid sequence at least 90%, 95%, 96%, 97%, 98%, or 99% identical to SEQ ID NO: 41 and the VL comprises an amino acid sequence at least 90%, 95%, 96%, 97%, 98%, or 99% identical to SEQ ID NO: 45;
(g) the VH comprises an amino acid sequence at least 90%, 95%, 96%, 97%, 98%, or 99% identical to SEQ ID NO: 49 and the VL comprises an amino acid sequence at least 90%, 95%, 96%, 97%, 98%, or 99% identical to SEQ ID NO: 53;
(h) the VH comprises an amino acid sequence at least 90%, 95%, 96%, 97%, 98%, or 99% identical to SEQ ID NO: 57 and the VL comprises an amino acid sequence at least 90%, 95%, 96%, 97%, 98%, or 99% identical to SEQ ID NO: 61;
(i) the VH comprises an amino acid sequence at least 90%, 95%, 96%, 97%, 98%, or 99% identical to SEQ ID NO: 65 and the VL comprises an amino acid sequence at least 90%, 95%, 96%, 97%, 98%, or 99% identical to SEQ ID NO: 69;
(j) the VH comprises an amino acid sequence at least 90%, 95%, 96%, 97%, 98%, or 99% identical to SEQ ID NO: 73 and the VL comprises an amino acid sequence at least 90%, 95%, 96%, 97%, 98%, or 99% identical to SEQ ID NO: 77;
(k) the VH comprises an amino acid sequence at least 90%, 95%, 96%, 97%, 98%, or 99% identical to SEQ ID NO: 81 and the VL comprises an amino acid sequence at least 90%, 95%, 96%, 97%, 98%, or 99% identical to SEQ ID NO: 85;
(l) the VH comprises an amino acid sequence at least 90%, 95%, 96%, 97%, 98%, or 99% identical to SEQ ID NO: 89 and the VL comprises an amino acid sequence at least 90%, 95%, 96%, 97%, 98%, or 99% identical to SEQ ID NO: 93;
(m) the VH comprises an amino acid sequence at least 90%, 95%, 96%, 97%, 98%, or 99% identical to SEQ ID NO: 97 and the VL comprises an amino acid sequence at least 90%, 95%, 96%, 97%, 98%, or 99% identical to SEQ ID NO: 101; or
(n) the VH comprises an amino acid sequence at least 90%, 95%, 96%, 97%, 98%, or 99% identical to SEQ ID NO: 105 and the VL comprises an amino acid sequence at least 90%, 95%, 96%, 97%, 98%, or 99% identical to SEQ ID NO: 109.

29. The compound of claim 19, wherein:
(a) the VH comprises an amino acid sequence of SEQ ID NO: 1 and the VL comprises an amino acid sequence of SEQ ID NO: 5;
(b) the VH comprises an amino acid sequence of SEQ ID NO: 9 and the VL comprises an amino acid sequence of SEQ ID NO: 13;
(c) the VH comprises an amino acid sequence of SEQ ID NO: 17 and the VL comprises an amino acid sequence of SEQ ID NO: 21;
(d) the VH comprises an amino acid sequence of SEQ ID NO: 25 and the VL comprises an amino acid sequence of SEQ ID NO: 29;
(e) the VH comprises an amino acid sequence of SEQ ID NO: 33 and the VL comprises an amino acid sequence of SEQ ID NO: 37;
(f) the VH comprises an amino acid sequence of SEQ ID NO: 41 and the VL comprises an amino acid sequence of SEQ ID NO: 45;
(g) the VH comprises an amino acid sequence of SEQ ID NO: 49 and the VL comprises an amino acid sequence of SEQ ID NO: 53;
(h) the VH comprises an amino acid sequence of SEQ ID NO: 57 and the VL comprises an amino acid sequence of SEQ ID NO: 61;
(i) the VH comprises an amino acid sequence of SEQ ID NO: 65 and the VL comprises an amino acid sequence of SEQ ID NO: 69;
(j) the VH comprises an amino acid sequence of SEQ ID NO: 73 and the VL comprises an amino acid sequence of SEQ ID NO: 77;
(k) the VH comprises an amino acid sequence of SEQ ID NO: 81 and the VL comprises an amino acid sequence of SEQ ID NO: 85;
(l) the VH comprises an amino acid sequence of SEQ ID NO: 89 and the VL comprises an amino acid sequence of SEQ ID NO: 93;
(m) the VH comprises an amino acid sequence of SEQ ID NO: 97 and the VL comprises an amino acid sequence of SEQ ID NO: 101; or
(n) the VH comprises an amino acid sequence of SEQ ID NO: 105 and the VL comprises an amino acid sequence of SEQ ID NO: 109.

30. The compound of any one of claims 1-29, wherein the compound is capable of binding to human CLDN18.2 with a K_{d} value of at most about 15 nM, at most about 12.5 nM, at most about 10 nM, at most about 7.5 nM, at most about 5 nM, or at most about 2.5 nM.

31. The compound of any one of claims 19-30, wherein the antibody or antigen binding fragment thereof is capable of binding to mouse CLDN18.2.

32. The compound of any one of claims 19-31, wherein the antibody or antigen binding fragment thereof is not capable of significantly binding to CLDN18.1.

33. The compound of any one of claims 19-32, wherein the antibody is a human antibody.

34. The compound of any one of claims 19-33, wherein the antigen binding fragment is a Fab, a F(ab′)2, a Fab′, a single-chain Fv (scFv), an Fv fragment, a Fd fragment, a diabody, or an isolated CDR or a combination of two or more isolated CDRs optionally joined by a linker.

35. The compound of any one of claims 19-34, wherein the antigen binding fragment is an scFv.

36. The compound of any one of claims 19-35, wherein the antibody or antigen binding fragment thereof comprises an antibody heavy chain constant region.

37. The compound of claim 36, wherein the heavy chain constant region is a human IgG heavy chain constant region.

38. The compound of claim 36 or 37, wherein the antibody heavy chain constant region is a human IgG4 heavy chain constant region.

39. The compound of claim 38, wherein the human IgG4 heavy chain constant region comprises a S228P mutation.

40. The compound of claim 1, wherein the compound comprises the following structure: 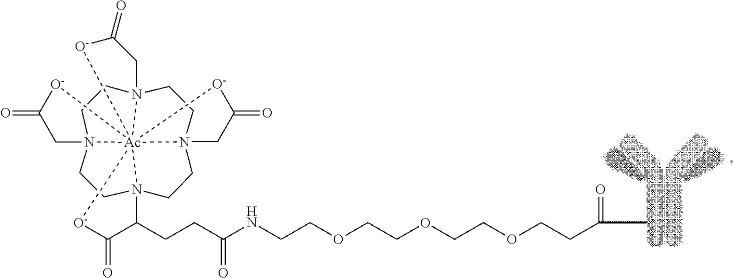
wherein [symbol] is an antibody or an antigen-binding fragment thereof that is capable of binding to CLDN 18.2 or a fragment thereof.

41. The compound of claim 41, wherein the antibody or an antigen-binding fragment thereof is linked to A-L- via the side-chain amino group of a lysine residue.

42. A pharmaceutical composition comprising a compound of any one of claims 1-41 and a pharmaceutically acceptable carrier, diluent, or excipient.

43. A method of radiation treatment planning and/or radiation treatment, the method comprising administering to a subject in need thereof a compound of any one of claims 1-41 or a pharmaceutical composition of claim 42.

44. A method of treating cancer comprising cells expressing CLDN 18.2, the method comprising administering to a subject in need thereof a first dose of a compound of any one of claims 1-41 or a pharmaceutical composition of claim 42 in an amount effective for radiation treatment planning, followed by administering subsequent doses of a compound of any one of claims 1-41 or the composition of claim 42 in a therapeutically effective amount.

45. The method of claim 44, wherein the compound or pharmaceutical composition administered in the first dose and the compound or pharmaceutical composition administered in a subsequent dose are the same.

46. The method of claim 44, wherein the compound or pharmaceutical composition administered in the first dose and the compound or pharmaceutical composition administered in a subsequent dose are different.

47. The method of any one of claims 43-46, wherein the cancer is esophageal cancer, gastric cancer, or pancreatic cancer.

48. The method of claim 47, wherein the cancer is esophageal adenocarcinoma.

49. The method of any one of claims 43-48, further comprising administering an antiproliferative agent, a radiation sensitizer, or an immunomodulatory agent.
